(19) United States
(12) Patent Application Publication
(10) Pub. No.: US 2026/0263268 A1
(43) Pub. Date: Sep. 10, 2026

(54) PROSTHETIC CAPSULAR DEVICES, SYSTEMS AND METHODS FOR DRUG DELIVERY

(71) Applicant: Omega Ophthalmics LLC, Versailles, KY (US)

(72) Inventors: Gary N. Wortz, Versailles, KY (US); Rick William Ifland, Versailles, KY (US)

(21) Appl. No.: 19/562,223

(22) Filed: Mar. 10, 2026

Related U.S. Application Data
(60) Provisional application No. 63/769,644, filed on Mar. 10, 2025.

(51) Int. Cl. A61F 9/00; A61F 2/00; A61F 2/16

(52) U.S. Cl. CPC A61F 9/0017; A61F 2/16; A61F 2002/0081; A61F 2250/0068

(57) ABSTRACT

The described technology pertains to ophthalmic prosthetics, specifically a prosthetic capsular device designed for placement into the natural capsular bag of the eye following lens removal. This device addresses challenges associated with invasive ocular drug delivery and inadequate drug localization by offering a platform for controlled, sustained release of pharmaceuticals. The housing structure includes an anterior portion, posterior portion, and lateral portion, which together form an internal cavity containing at least one chamber for the slow release of pharmaceuticals. The device may feature porosity to facilitate drug diffusion, supports intraocular lenses (IOLs), and is designed to be self-expandable and deformable for ease of insertion. Furthermore, the device is compatible with flexible drug-eluting devices and enables repeated drug delivery throughout a patient's lifetime, thereby mitigating risks of inflammation and complications associated with traditional methods.

### PRIORITY APPLICATIONS

This application claims priority to U.S. Provisional Patent Application No. 63/769,644, filed Mar. 10, 2025, which is hereby incorporated by reference in its entirety and for all purposes. Any and all applications for which a foreign or domestic priority claim is identified in the Application Data Sheet as filed with the present application are hereby incorporated by reference under 37 CFR 1.57.

### BACKGROUND

### Field

The embodiments of the disclosure generally relate to ophthalmic prosthetics, and more particularly to devices, systems, and methods for a prosthetic apparatus for insertion into the eye configured for drug delivery.

### Description of the Related Art

Cataract surgery is one of the most successfully and most frequently performed surgical procedures in the United States. Each year, millions of people achieve a dramatic improvement in their visual function thanks to this procedure. With the increasing proportion of the U.S. population reaching their retirement years, there is expected to be an almost doubling of the demand for cataract surgery over the next twenty years from 3.3 million to over 6 million annually. In response to the increased demand, more ophthalmologists may be trained and certified to perform cataract surgery, and each trained and certified ophthalmologist may perform more cataract surgeries each year.

### SUMMARY

The field of ophthalmic prosthetics has long sought effective solutions for drug delivery within the eye, particularly in the context of post-surgical care and chronic conditions requiring sustained medication. Conventional approaches, such as intravitreal injections, while effective in delivering drugs to the posterior segment of the eye, are fraught with limitations. These include the need for frequent invasive procedures, potential damage to ocular structures such as the lens capsule, increased intraocular pressure, and risks of complications like retinal detachment and endophthalmitis. Further, the anterior segment of the eye has only recently been considered for drug delivery, but existing methods suffer from poor control of drug localization, leading to adverse effects such as corneal endothelial damage and iris atrophy. These challenges underscore the need for a more controlled, less invasive, and sustained drug delivery mechanism that minimizes risks while maximizing therapeutic efficacy.

The present disclosure addresses these limitations by introducing a prosthetic capsular device configured for insertion into the natural capsular bag of the eye. This prosthetic device creates a dedicated space within the eye for housing drug delivery systems, enabling controlled and sustained release of pharmaceuticals directly to the targeted ocular tissues. Unlike conventional methods, the prosthetic capsular device eliminates the need for repeated injections by allowing for the insertion of drug delivery devices that can dissolve, partially dissolve, or elute drugs over time. The prosthetic capsular device is designed to position these drug delivery systems peripherally to the visual pathway, ensuring that visual function remains unobstructed. Additionally, the prosthetic capsular device can support intraocular lenses (IOLs), further enhancing its applicability in post-cataract surgery scenarios.

The described concept utilizes advanced material properties and structural design to enhance drug delivery. For example, the device may be impregnated with active pharmaceutical agents during manufacturing or coated with drugs after production, enabling gradual diffusion into the eye. Alternatively, the device may incorporate bioerodible drug delivery systems that release medication in a controlled manner. The self-expandable housing of the device promotes long-term stability within the natural capsular bag, while its specialized openings support the insertion and removal of drug delivery systems throughout the patient's lifetime. By addressing limitations of current drug delivery methods and offering a flexible platform for sustained ocular therapy, this approach marks a notable progression in ophthalmic prosthetics and drug delivery technology.

According to an aspect of the present disclosure, a system for intraocular drug delivery comprises a prosthetic capsular device configured to be inserted into a natural capsular bag of an eye after removal of a lens, the prosthetic capsular device comprising a housing structure having an anterior portion with an anterior opening, a posterior portion with a posterior opening, and a lateral portion interposed between the anterior portion and the posterior portion, the anterior portion, posterior portion, and lateral portion together defining an internal cavity. The system further comprises a drug delivery capsule configured to be received within the internal cavity of the prosthetic capsular device, the drug delivery capsule comprising a posterior surface and an anterior surface meeting at an outer end of the capsule, an interior surface having a shape of a section of a radial surface of a cylinder and being concave, a radial surface extending from an inner end to the outer end of the capsule, an inner cavity configured to receive a drug, one or more diffusion openings extending from an exterior surface of the capsule to the inner cavity and configured to permit the drug to elute from the inner cavity into the interior of the prosthetic capsular device, and a slit extending from the exterior surface of the capsule to the inner cavity and configured to permit introduction of the drug into the inner cavity, wherein the drug delivery capsule is sized and shaped such that the concave interior surface of the capsule conforms at least in part to an interior surface of the housing structure when the capsule is disposed within the internal cavity, and wherein the prosthetic capsular device and the drug delivery capsule together are configured to position the drug in a location peripherally of a visual axis of the eye and to dispense the drug from within the natural capsular bag over an extended period of time.

According to some embodiments, the prosthetic capsular device may further comprise a posterior flange disposed on a posterior interior surface of the housing structure and an anterior flange disposed on an anterior interior surface of the housing structure, the posterior flange and the anterior flange extending toward one another to retain the drug delivery capsule within the internal cavity.

In some embodiments, the posterior flange and the anterior flange can be configured to meet along a middle plane of the prosthetic capsular device.

In some embodiments, the posterior flange and the anterior flange might be separated by a gap configured to permit insertion and removal of the drug delivery capsule.

According to some embodiments, the one or more diffusion openings may be disposed on the concave interior surface of the drug delivery capsule.

According to some embodiments, the one or more diffusion openings may be disposed on the radial surface of the drug delivery capsule.

In some embodiments, the slit may be disposed on the concave interior surface of the drug delivery capsule and can be configured to receive an instrument for injecting the drug into the inner cavity.

According to some embodiments, the inner cavity of the drug delivery capsule may be pre-filled with the drug during manufacture.

In some embodiments, the drug may comprise at least one of a solid drug, a gel drug, a polymerized drug, or a liquid drug.

According to some embodiments, the internal cavity of the prosthetic capsular device may be configured to receive a plurality of said drug delivery capsules.

In some embodiments, at least a portion of the prosthetic capsular device, the drug delivery capsule, or both may be formed from crosslinked polyisobutylene.

According to some embodiments, the prosthetic capsular device may be substantially symmetrical about a plane extending through a midpoint between the anterior portion and the posterior portion.

In some embodiments, the prosthetic capsular device may be asymmetrical such that the anterior portion has a different shape, contour, angle, thickness, or radial profile than the posterior portion.

According to an aspect of the present disclosure, an intraocular drug delivery device comprises an annular body having an interior wall and a lateral outsidewall, the annular body defining an interior chamber configured to contain a pharmaceutical, wherein the interior wall comprises a plurality of pores extending therethrough and configured to permit the pharmaceutical to diffuse from the interior chamber into an eye, and wherein the interior wall further comprises a fill port in fluid communication with the interior chamber and configured to receive the pharmaceutical.

According to some embodiments, the intraocular drug delivery device may be configured to be inserted into a prosthetic capsular device.

In some embodiments, the fill port may be disposed on the interior wall and may be circular.

According to some embodiments, the fill port may be sized to receive a needle.

In some embodiments, the lateral outside wall may comprise a slit, opening, or cut extending from an exterior surface of the annular body to the interior chamber.

According to some embodiments, the slit, opening, or cut may have a wave-like shape.

In some embodiments, the slit, opening, or cut may be sealed with silicone or an adhesive after formation of the interior chamber.

### BRIEF DESCRIPTION OF THE DRAWINGS

In the drawings, which are not necessarily to scale, like numerals may describe similar components in different views. Like numerals having different letter suffices may represent different instances of similar components. The drawings illustrate generally, by way of example, but not by way of limitation, various embodiments discussed in the present document.

FIGS. 1A and 1B illustrate an example prosthetic capsular device according to some embodiments herein. FIG. 1A is a transparent perspective view and FIG. 1B is a cross-sectional view thereof.

FIGS. 2A and 2B illustrate another example prosthetic capsular device according to some embodiments herein. FIG. 2A is a perspective view and FIG. 2B illustrates various additional views thereof.

FIGS. 3A-3D illustrate another example prosthetic capsular device according to some embodiments herein. FIG. 3A is a perspective view, FIG. 3B is an anterior view, FIG. 3C is a cross-sectional side view, and FIG. 3D is a side view.

FIGS. 4A-4D illustrate an example drug delivery capsule according to some embodiments herein. FIGS. 4A and 4B illustrate first and second perspective views, FIG. 4C illustrates a cross-sectional view, and FIG. 4D is a side view.

FIGS. 5A-5C illustrate an example prosthetic capsular device according to some embodiments herein.

FIGS. 5D-5F illustrate an example prosthetic capsular device according to some embodiments herein.

FIGS. 5G-5I illustrate an example prosthetic capsular device according to some embodiments herein.

FIGS. 6A-6C illustrate an embodiment of an ocular implant with multiple chambers that is two distinct ends.

FIGS. 7A-7B illustrate an example prosthetic capsular device according to some embodiments herein.

FIG. 7C illustrates a cross-section of an embodiment a flexible insert in the prosthetic capsular device according to some embodiments herein.

FIG. 8A illustrates an embodiment of an ocular implant with multiple chambers and ports.

FIG. 8B illustrate an embodiment of a port according to some embodiments herein.

FIGS. 9A-9C illustrate an example prosthetic capsular device according to some embodiments herein.

FIGS. 10A-10H illustrate another example prosthetic capsular device according to some embodiments herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be conducted in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present technology.

All patents, patent applications, and other documents referred to in this application are incorporated by reference herein in their entirety.

In addition to the increase in demand for cataract surgery, technological advances have increased patient expectations for the surgery. The procedure takes a short amount of time to perform, and patients expect quick recovery of visual function. Patients are also asking their ophthalmologist to give them the restoration of more youthful vision without glasses through the use multifocal intraocular lenses, extended depth of focus lenses, accommodating lenses, other presbyopia correcting lenses, toric lenses, and monovision, to name a few. Despite accurate preoperative measurements and excellent surgical technique, post-surgical outcomes may vary due to undesirable physiological interaction with surgical implants.

The intravitreal space has been utilized widely over the past 30 plus years for injection of steroids for treatment of inflammatory and vascular disease processes. In the past 15 years or so, the intravitreal space has been widely utilized for treatment of wet age-related macular degeneration and other vascular proliferative retinal diseases with injection of Anti-Vegf monoclonal antibodies, dexamethasone, and fluocinolone acetanide and other drugs.

Drug delivery in the vitreous cavity advantageously allows for a large potential volume of drugs to be placed in close proximity to the retina. However, drug delivery in the vitreous cavity requires an injection, usually monthly or some other period. The medications are sometimes opaque creating a barrier to light being received by the retina and/or loss of vision. Intravitreal injections can damage the natural lens capsule and lens, increase intraocular pressure, and/or can cause retinal detachment and endophthalmitis or other damage to the eye tissue.

The anterior segment of the eye has only recently been regarded as a possible target for drug delivery. Recently drugs such as dexamethasone and bimatorprost have been FDA approved for the treatment of post-operative inflammation and glaucoma, respectively. While both of these treatment options can be effective, each is plagued by having poor control of drug location. When a drug is placed in the anterior segment it can cause damage if it migrates adjacent to the corneal endothelium. Similarly, iris atrophy has been noted in patients with certain drugs being in prolonged contact with the iris. Unlike the posterior segment (vitreous cavity), controlling the location of controlled release drug implants plays a significant role in preventing complications.

Accordingly, it can be advantageous to utilize an ophthalmic device configured to continuously deliver drugs to the eye tissue thereby eliminating the need for periodic injections into the eye and avoiding the negative consequences of such injections. In some embodiments, such an ophthalmic device can comprise a prosthetic capsular device for insertion into the natural capsular bag, and in some embodiments, the prosthetic capsular device can be configured to create a space or void or volume within the eye for receiving drug delivery devices for placement in the eye.

In some embodiments, the prosthetic capsular device can also be configured to support one or more intraocular lenses (IOLs). A drug delivery device can be inserted into the prosthetic capsular device, and in some embodiments be positioned peripherally to the visual pathway such that the drug delivery device does not or does not materially or does not substantially obstruct, interfere, or block the visual pathway. In some environments, the drug delivery device is configured to dissolve or partially dissolve, or otherwise change over time, thereby releasing into the eye at least a portion of the drugs in the drug delivery device. In some embodiment, the prosthetic capsular device may receive one or more secondary drug delivery devices, for example, after a first drug delivery device dissolves or after substantially all the drugs in the drug delivery device has been released into the eye, or for example, simultaneous or subsequent with the first drug delivery device in order to release multiple drugs into the eye at the same time.

In some embodiments, a drug or drug delivery device can be inserted into the housing/body of the device, such that the prosthetic capsular device is impregnated with an active pharmaceutical agent during the molding process. In some embodiments, the prosthetic capsule can be coated either on the interior, the exterior, or both with a drug after the molding process such that the drug dissolves off of the surface of the prosthetic capsule. In some embodiments, the prosthetic capsule is placed into a solution containing active pharmaceutical agents at some point after molding (either before packaging, or afterward such as immediately prior to surgery) such that it soaks up drug into the silicone pores, through which drug can dissolve into the eye over time. In some embodiments, the drug delivery device is composed of a biocompatible flexible non-absorbable material with active pharmaceutical agent inside of it, with an opening or multiple openings allowing for the controlled release of the drug. In such embodiments, the drug delivery device could be explanted from the eye once the active pharmaceutical agent has substantially dissolved and a new device could be inserted. This process could take place over the patient's lifetime for extant treatment of disease processes because the housing of the prosthetic capsular device is configured to allow for the insertion and removal of the drug delivery device or allow for the insertion of new drugs.

In some embodiments, the housing/body of the prosthetic capsular device is configured for the insertion of new drug delivery devices or drugs over years and decades because the housing of the prosthetic capsular device is configured to remain in a self-expanded state while positioned in the natural capsular bag, and the housing of the device comprises an opening that is configured to remain open while positioned in the natural capsular bag, and the opening allows for drug delivery devices and/or drugs to be inserted into the interior of the housing of the prosthetic capsular device. In some embodiments, the drug delivery device can be bio erodible. In some embodiments, the drug delivery device may release one or more drug into the natural capsular bag. In some embodiments, the drug delivery device can be configured to comprise a substantially linear elongated shape configuration. In some embodiments, the substantially linear elongated shape configuration enables the drug delivery device to be easily inserted into a delivery cannula or trocar or injector in order to position the drug delivery device within the prosthetic capsular device. In some embodiments, the substantially linear elongated shaped drug delivery device is configured to bend within the prosthetic capsular device. In some embodiments, the bending of the linear elongated shaped drug delivery device allows the drug delivery device to be positioned in the peripheral area of the prosthetic capsular device in order to avoid, obstruct or otherwise block the visual pathway of the eye. In some embodiments, the substantially linear elongated shaped drug delivery device can comprise end portions that are substantially curved whereas a mid-portion of the drug delivery device is substantially linear.

In some embodiments, the drug delivery device can be configured to comprise a substantially curved shaped configuration. In some embodiments, the drug delivery device can be configured to comprise a bias such that the drug delivery device takes on a curved shaped configuration. In some embodiments, the curved shaped drug delivery device can be straightened into a more linear configuration in order for the drug delivery device to be inserted through an insertion cannula or trocar or injector for implantation into the prosthetic capsular device. In some embodiments, after the curved shaped drug delivery device is positioned into the prosthetic capsular device, the inherent bias of the drug delivery device allows the drug delivery device to take on a curved shape within the prosthetic capsular device. In some embodiments, the bias of the curved shaped drug delivery device enables the drug delivery device to position itself into the peripheral area of the prosthetic capsular device in order to avoid or interfere or obstruct with the visual pathway of the eye. In some embodiments, the biased drug delivery device is configured to self-expand within the prosthetic capsular device in such a way as to take on a curved shape. In some embodiments, the drug delivery device can bend and/or self-expand from a substantially straight configuration to a substantially ring-shaped configuration. In some embodiments, the drug delivery device can comprise a substantially cylindrical shape. In some embodiments, the drug delivery device can comprise a profile shape. In some embodiments, the profile shape can be substantially circular shaped, oval shaped, elliptical shaped, diamond shaped, triangle shaped, rectangle shaped, pentagon shaped, hexagon shaped, octagon shaped, and/or any other shape. In some embodiments, the drug delivery device comprises substantially smooth and/or rounded edges, and in some embodiments, the smooth and/or rounded edges prevent scraping against the sidewalls of the prosthetic capsular device. In some embodiments, the prosthetic capsular device comprises a groove portion in the interior of the housing of the prosthetic capsular device, wherein the groove portion is configured to receive and/or retain the drug delivery device once the drug delivery device has been inserted into the prosthetic capsular device and self-expands and/or returns to drug delivery device's biased curved and/or ring-shaped configuration.

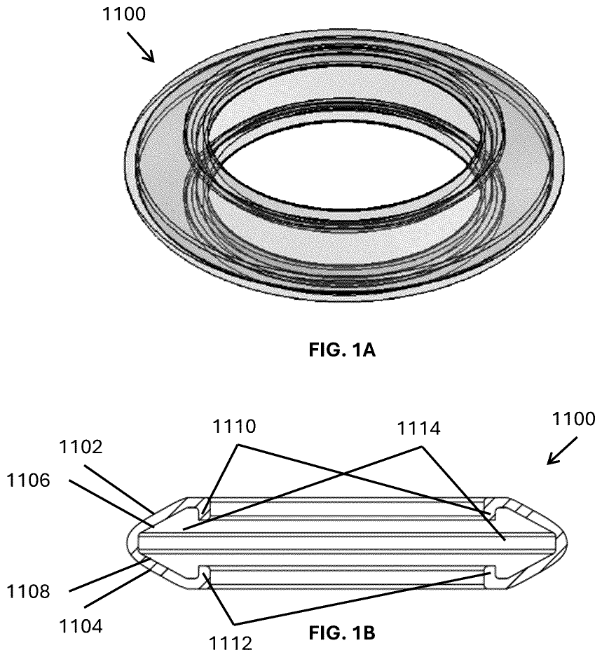

FIGS. 1A and 1B illustrate an example prosthetic capsular device 1100 according to some embodiments herein. FIG. 1A is a transparent perspective view and FIG. 1B is a cross-sectional view thereof.

In some embodiments, the device 1100 includes features described with respect to the devices described in U.S. Pat. No. 9,358,103, which is hereby incorporated by reference in its entirety, or modifications thereof.

In some embodiments, the device 1100 can comprise a posterior exterior surface 1102 disposed opposite from an anterior exterior surface 1104. The posterior exterior surface 1102 can meet the anterior exterior surface 1104 at an outermost circumference of the device 1100. The posterior exterior surface 1102 can be separated from the anterior exterior surface 1104 by an outer sidewall disposed at an outermost circumference of the device 1100.

In some embodiments, the posterior exterior surface 1102 can have a slope such that the innermost portion of the posterior exterior surface 1102 extends in a posterior direction. The posterior exterior surface 1102 can have a slope such that the outermost portion of the posterior exterior surface 1102 extends in an anterior direction. In some embodiments, the posterior exterior surface 1102 can be shaped to fit flush in a cone (i.e., the posterior exterior surface 1102 can be shaped without curvature in the posterior-anterior directions). The posterior exterior surface 1102 can be convex (i.e., the posterior exterior surface 1102 can be shaped with positive curvature in the posterior-anterior directions). The posterior exterior surface 1102 can be concave (i.e., the posterior exterior surface 1102 can be shaped with negative curvature in the posterior-anterior directions). The posterior exterior surface 1102 can have multiple segments, with different segments having positive, negative, or no curvature in the posterior-anterior directions.

In some embodiments, the anterior exterior surface 1104 can have a slope such that the innermost portion of the anterior exterior surface 1104 extends in an anterior direction. The anterior exterior surface 1104 can have a slope such that the outermost portion of the anterior exterior surface 1104 extends in a posterior direction. In some embodiments, the anterior exterior surface 1104 can be shaped to fit flush in a cone (i.e., the anterior exterior surface 1104 can be shaped without curvature in the posterior-anterior directions). The anterior exterior surface 1104 can be convex (i.e., the anterior exterior surface 1104 can be shaped with positive curvature in the posterior-anterior directions). The anterior exterior surface 1104 can be concave (i.e., the anterior exterior surface 1104 can be shaped with negative curvature in the posterior-anterior directions). The anterior exterior surface 1104 can have multiple segments, with different segments having positive, negative, or no curvature in the posterior-anterior directions.

In some embodiments, the posterior exterior surface 1102 can extend from an outermost circumference of the device 1100 to an innermost circumference of the device. In some embodiments, the anterior exterior surface 1104 can extend from an outermost circumference of the device 1100 to an innermost circumference of the device. The outermost circumference of the device 1100 can have a diameter of between 130% and 200% of the diameter of the innermost circumference of the device 1100. The outermost circumference of the device 1100 can have a diameter of about 162% of the diameter of the innermost circumference of the device 1100. The outermost circumference of the device 1100 can have a diameter of about 130%, 135%, 140%, 145%, 150%, 155%, 160%, 165%, 170%, 175%, 180%, 185%, 190%, 195%, or 200% of the diameter of the innermost circumference of the device 1100.

In some embodiments, the device 1100 can comprise a posterior interior surface 1106 disposed opposite from an anterior interior surface 1108. The posterior interior surface 1106 can meet the anterior interior surface 1108 at an outer circumference of the device 1100. The posterior interior surface 1106 can be separated from the anterior interior surface 1108 by an outer sidewall disposed at an outer circumference of the device 1100. The posterior interior surface 1106 can be parallel to the posterior exterior surface 1102 (i.e., the posterior portion of the device 1100 can have a constant thickness). The posterior interior surface 1106 can have varying distances from the posterior exterior surface 1102 (i.e., the posterior portion of the device 1100 can have a variable thickness). The anterior interior surface 1108 can be parallel to the anterior exterior surface 1104 (i.e., the anterior portion of the device 1100 can have a constant thickness). The anterior interior surface 1108 can have varying distances from the anterior exterior surface 1104 (i.e., the anterior portion of the device 1100 can have a variable thickness).

In some embodiments, the posterior interior surface 1106 can have a slope such that the innermost portion of the posterior interior surface 1106 extends in a posterior direction. The posterior interior surface 1106 can have a slope such that the outermost portion of the posterior interior surface 1106 extends in an anterior direction. In some embodiments, the posterior interior surface 1106 can be shaped to fit flush over a cone (i.e., the posterior interior surface 1106 can be shaped without curvature in the posterior-anterior directions). The posterior interior surface 1106 can be convex (i.e., the posterior interior surface 1106 can be shaped with positive curvature in the posterior-anterior directions). The posterior interior surface 1106 can be concave (i.e., the posterior interior surface 1106 can be shaped with negative curvature in the posterior-anterior directions). The posterior interior surface 1106 can have multiple segments, with different segments having positive, negative, or no curvature in the posterior-anterior directions.

In some embodiments, the anterior interior surface 1108 can have a slope such that the innermost portion of the anterior interior surface 1108 extends in an anterior direction. The anterior interior surface 1108 can have a slope such that the outermost portion of the anterior interior surface 1108 extends in a posterior direction. In some embodiments, the anterior interior surface 1108 can be shaped to fit flush over a cone (i.e., the anterior interior surface 1108 can be shaped without curvature in the posterior-anterior directions). The anterior interior surface 1108 can be convex (i.e., the anterior interior surface 1108 can be shaped with positive curvature in the posterior-anterior directions). The anterior interior surface 1108 can be concave (i.e., the anterior interior surface 1108 can be shaped with negative curvature in the posterior-anterior directions). The anterior interior surface 1108 can have multiple segments, with different segments having positive, negative, or no curvature in the posterior-anterior directions.

In some embodiments, the device 1100 can comprise an interior cavity 1114 disposed between an outer circumference of the device 1100 and an inner circumference of the device 1100. The interior cavity 1114 can be disposed in an anterior direction from the posterior internal surface 1106. The internal cavity 1114 can be adjacent to the posterior internal surface 1106. The interior cavity 1114 can be disposed in a posterior direction from the anterior internal surface 1108. The internal cavity 1114 can be adjacent to the anterior internal surface 1108. The internal cavity 1114 can extend along an entire circumference of the device (i.e., the internal cavity 1114 can have an angular width of 360°). The internal cavity 1114 can extend along a portion of a circumference of the device (i.e., the internal cavity 1114 can have an angular width less than 360°). The internal cavity 1114 can comprise more than one cavity segment disposed along a circumference of the device.

Device 1100 can be configured to comprise one or more intraocular lenses, electronic devices, or other intraocular devices held within the internal cavity 1114. The IOLs may comprise any and all lens powers and designs that are currently known in the art of intraocular lenses, including, but not limited to: spherical, aspheric, wavefront, convex, concave, extended depth of focus, pinhole or small aperture, multifocal (diffractive, refractive, zonal), toric, accommodative, ultraviolet (UV) filtering, diffractive chromatic aberration reducing lenses, light adjustable lenses (ultraviolet light adjustable, femtosecond phase wrapping), and optical powers ranging from any positive diopter value (e.g., including +35 D and above) to any negative diopter value (e.g., including −35 D and below).

Further, in certain embodiments, device 1100 includes one or more additional features. For example, device 1100 can comprise a generally lenticular or lens-like shape as opposed to a box-like design. In other words, the generally shape of the device 1100 can be more like the shape of a natural lens. Risks of negative and/or positive dysphotopsia can be reduced due to the generally lenticular shape of the device 1100. Negative dysphotopsia is a widespread problem in cataract surgery, generally described by patients as a temporal dark crescent in their vision and is believed to occur either due to the optical phenomenon known as total internal reflection or by obstruction of light. This can occur either at the junction of the optic edge and the empty collapsed surrounding capsule forming a relatively planar surface, or due to the capsule overlapping a portion of the optic, most commonly the nasal aspect. In embodiments in which the implantable device 1100 comprises an overall lens-like configuration, the capsule can be held open, preventing a relatively planar surface from being formed by fusion of the posterior and anterior capsule. More specifically, when light hits a curvilinear slice of the device 1100, which can be made from silicone for example, it may travel through the curvilinear slice instead of bouncing off and causing a negative shadow as it generally would for flat surfaces. This may be especially true in the horizontal meridian across the 180-degree plane. As such, in some embodiments, device 1100 does not comprise any flat edges or surfaces. In other words, every surface of the device 1100 can be curvilinear. Flat optical surfaces can promote total internal reflection and are not found in the natural human lens or lens capsule in the native state. One goal of some of the embodiments described herein is to reduce negative dysphotopsias by not having any flat optical surfaces. Certain embodiments may have additional features such as an opaque or translucent tint. This may further enhance the reduction of positive dysphotopsias by blocking stray light that could be reflected off of the IOL border or haptic edges. This could also function as an artificial iris of sorts, depending on the color and opacity of the tint, blocking light that could be transmitted through an iris transillumination defect, a traumatically altered iris, or a surgical peripheral iridotomy, likewise preventing positive dysphotopsias and glare.

In some embodiments, substantially the whole device 1100 can comprise silicone and/or a soft silicone polymer. In addition, in certain embodiments, substantially the whole device 1100 can comprise a flexible and/or elastic material. As such, device 1100 can be foldable or collapsible for implantation into the eye through a small incision. Once inserted into the eye, device 1100 can naturally unfold and self-expand into its expanded configuration as illustrated in FIG. 1A. Device 1100 can comprise one or more capsular areas. The one or more capsular areas can be adapted to receive and/or hold an IOL. In some embodiments, one or more sidewalls can comprise a concave shape. For example, the interior surface of the one or more sidewalls can form a cavity. The cavity can be configured to hold an IOL, for example.

In some embodiments, device 1100 comprises a single-molded design. In other words, the whole device 1100, or substantially the whole device 1100 can be molded from a single piece of material. For example, in some embodiments, substantially the whole device 1100 can be molded of silicone using a silicone compression mold. In other embodiments, the device 1100 or any portion thereof can be manufactured by 3D laser cutting, two photon lithography, additive manufacturing, 3D printing, compression molding, and/or any combination of the aforementioned manufacturing processes or others.

In some embodiments, the device 1100 can be inserted through an incision between about 1.5 mm and about 3 mm (e.g., about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, ranges between such values, etc.).

Further, in some embodiments, a length of a major axis of the device 1100 or a length measured from the outermost end of one sidewall to the outermost end of another sidewall along a major axis of the device 1100 can be about 9.65 mm. In other embodiments, the length of the major axis of the device 1100 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1100 may comprise an outermost diameter of the device 100.

In some embodiments, the thickness of silicone or other material of the device 1100 can be about 0.25 mm. In certain embodiments, the thickness of silicone or other material of the device 1100 can be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or within a range defined by two of the aforementioned values.

In some embodiments, the thickness of the silicone or other material of the device 1100 varies depending on the portion of the device 1100. In other words, some portions of device 1100 can be made of thinner materials while other portions of device 1100 can be made of thicker materials. For example, certain portions of the device that provide support to the anterior portion of the device 1100 may be made with thicker materials for added support.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an innermost diameter, which can be a diameter of an anterior opening and/or a posterior opening.

In some embodiments, the shape and size of the device 1100 may minimize anterior, posterior, and/or radial protrusion into the natural capsular bag relative to previously used capsular devices. In some embodiments, the device 1100 may be smaller in certain dimensions especially towards the anterior and periphery of the device. In some embodiments, sharp and/or straight edges or sides may not be present in the device 1100 to reduce friction between the device 1100 and the posterior aspect of the iris of an eye. In some embodiments, the smaller size, decrease in anterior, posterior, and/or radial protrusion into the natural capsular bag, and smoothened or curved edges, may result in the device 1100 having an enhanced biocompatibility profile and/or may reduce inflammation caused by the device in the eye. In some embodiments, the unique shape and design of device 1100 may result in a decrease and/or elimination of inflammation of the eye (e.g., anterior of the eye) upon insertion of the device. In some embodiments, the decrease and/or elimination of post-insertion inflammation resulting from the shape of the device 1100 may result in a decrease and/or elimination of the need for post-operative anti-inflammatory medications such as, e.g., steroids or nonsteroidal anti-inflammatory drugs (NSAIDs). It may also result in a decrease and/or elimination of device removals and/or replacements, which may be needed if inflammation cannot be reduced or removed.

The device 1100 can be self-expandable to keep the capsule open. The device 1100 can comprise at least three different planes. For example, a first plane can correspond with the posterior opening or end of the device, where an IOL can be attached. A second plane can correspond with the anterior opening or end of the device, where another refractive surface or IOL can be attached. In some embodiments, device 1100 comprises a symmetrical device such that the anterior opening and posterior opening are determined by the device 1100 position in the eye. A third plane can be positioned in between the posterior end and the anterior end, for example, along a ridge formed in the central portion, where another an IOL can be attached. In some embodiments, the central portion may comprise a continuous lateral portion interposed between the anterior portion and the posterior portion. In some embodiments, the continuous lateral portion protrudes radially beyond the anterior portion and the posterior portion. In some embodiments, the continuous lateral portion fully encloses a lateral side of the housing structure, wherein an internal cavity of the continuous lateral portion forms a groove for containing an IOL. In some embodiments, the central portion may comprise a continuous lateral portion comprising an exterior surface comprising a rounded bulge, the rounded bulge extending radially beyond the anterior portion and the posterior portion. In some embodiments, the continuous lateral portion comprises an interior surface comprising a groove or ridge, wherein at least a portion of the interior surface is formed at an acute angle or an obtuse angle relative to the anterior portion and the posterior portion.

In some embodiments, a prosthetic capsular device configured to be inserted in a natural capsular bag of an eye after removal of a lens can comprise a housing structure 1100 capable of containing one or more intraocular devices and/or refractive surfaces. In particular, the housing structure can comprise an anterior side, wherein the anterior side comprises an anterior opening that can be elliptical, circular, arcuate, triangular, rectangular, polygonal, or otherwise shaped as shown in the provided Figures, wherein the anterior opening is capable of allowing at least one of insertion, removal, or replacement of an intraocular lens device, and wherein the anterior opening is further configured to be coupled to a lens to cover the anterior opening; a posterior side, wherein the posterior side comprises an posterior opening that can be elliptical, circular, arcuate, triangular, rectangular, polygonal, or otherwise shaped as shown in the provided Figures, wherein the posterior opening is capable of allowing at least one of insertion, removal, or replacement of an intraocular lens or device, and wherein the posterior opening is further configured to be coupled to an intraocular lens or device to cover the posterior opening; and a continuous lateral portion interposed between the anterior portion and the posterior portion, wherein the continuous lateral portion protrudes radially beyond the anterior portion and the posterior portion, wherein the continuous lateral portion fully encloses a lateral side of the housing structure, wherein an internal cavity of the continuous lateral portion forms a groove or ridge for containing an intraocular lens or device within, for example, an anterior portion of the device. The continuous lateral portion may not have any openings, for example along the lateral portion of the device in some embodiments. The housing structure 1100 can be symmetrical over a plane at a midpoint of the continuous lateral portion between the anterior portion and the posterior portion.

In some embodiments, the device 1100 includes flanges 1110, 1112 disposed on the interior side of the device 1100. The device can include a posterior flange 1110 disposed on the posterior interior surface 1106. The device can include an anterior flange 1112 disposed on the anterior interior surface 1108. The posterior flange 1110 can extend toward the anterior flange 1112. In some embodiments, the flanges 1110, 1112 can be configured to meet. In some embodiments, the flanges 1110, 1112 can be configured to meet along a middle plane of the device 1100. In some embodiments, the flanges 1110, 1112 can be separated by a gap. In some embodiments, the flanges 1110, 1112 can be configured to meet along a complementary surface. In some embodiments, the flanges 1110, 1112 can be configured to meet along an irregular complementary surface. In some embodiments, the flanges 1110, 1112 can be configured to meet along a complementary surface having a wave-like profile. In some embodiments, the flanges 1110, 1112 can overlap.

In some embodiments, the posterior flange 1110 can have a length between about 0.5 and about 5 mm. The posterior flange 1110 can have a length of about 0.5 mm. The posterior flange 1110 can have a length of about 1 mm. The posterior flange 1110 can have a length of about 1.5 mm. The posterior flange 1110 can have a length of about 2 mm. The posterior flange 1110 can have a length of about 2.5 mm. The posterior flange 1110 can have a length of about 3 mm. The posterior flange 1110 can have a length of about 3.5 mm. The posterior flange 1110 can have a length of about 4 mm. The posterior flange 1110 can have a length of about 4.5 mm. The posterior flange 1110 can have a length of about 5 mm.

In some embodiments, the posterior flange 1110 can have a length that extends between about 10% and about 90% of the total thickness of the device 1000 in the posterior-anterior directions. The posterior flange 1110 can have a length that extends about 10% of the total thickness of the device 1000 in the posterior-anterior directions. The posterior flange 1110 can have a length that extends about 15% of the total thickness of the device 1000 in the posterior-anterior directions. The posterior flange 1110 can have a length that extends about 20% of the total thickness of the device 1000 in the posterior-anterior directions. The posterior flange 1110 can have a length that extends about 25% of the total thickness of the device 1000 in the posterior-anterior directions. The posterior flange 1110 can have a length that extends about 30% of the total thickness of the device 1000 in the posterior-anterior directions. The posterior flange 1110 can have a length that extends about 35% of the total thickness of the device 1000 in the posterior-anterior directions. The posterior flange 1110 can have a length that extends about 40% of the total thickness of the device 1000 in the posterior-anterior directions. The posterior flange 1110 can have a length that extends about 45% of the total thickness of the device 1000 in the posterior-anterior directions. The posterior flange 1110 can have a length that extends about 50% of the total thickness of the device 1000 in the posterior-anterior directions. The posterior flange 1110 can have a length that extends about 55% of the total thickness of the device 1000 in the posterior-anterior directions. The posterior flange 1110 can have a length that extends about 60% of the total thickness of the device 1000 in the posterior-anterior directions. The posterior flange 1110 can have a length that extends about 65% of the total thickness of the device 1000 in the posterior-anterior directions. The posterior flange 1110 can have a length that extends about 70% of the total thickness of the device 1000 in the posterior-anterior directions. The posterior flange 1110 can have a length that extends about 75% of the total thickness of the device 1000 in the posterior-anterior directions. The posterior flange 1110 can have a length that extends about 80% of the total thickness of the device 1000 in the posterior-anterior directions. The posterior flange 1110 can have a length that extends about 85% of the total thickness of the device 1000 in the posterior-anterior directions. The posterior flange 1110 can have a length that extends about 90% of the total thickness of the device 1000 in the posterior-anterior directions.

In some embodiments, the anterior flange 1112 can have a length between about 0.5 and about 5 mm. The anterior flange 1112 can have a length of about 0.5 mm. The anterior flange 1112 can have a length of about 1 mm. The anterior flange 1112 can have a length of about 1.5 mm. The anterior flange 1112 can have a length of about 2 mm. The anterior flange 1112 can have a length of about 2.5 mm. The anterior flange 1112 can have a length of about 3 mm. The anterior flange 1112 can have a length of about 3.5 mm. The anterior flange 1112 can have a length of about 4 mm. The anterior flange 1112 can have a length of about 4.5 mm. The anterior flange 1112 can have a length of about 5 mm.

In some embodiments, the anterior flange 1112 can have a length that extends between about 10% and about 90% of the total thickness of the device 1000 in the posterior-anterior directions. The anterior flange 1112 can have a length that extends about 10% of the total thickness of the device 1000 in the posterior-anterior directions. The anterior flange 1112 can have a length that extends about 15% of the total thickness of the device 1000 in the posterior-anterior directions. The anterior flange 1112 can have a length that extends about 20% of the total thickness of the device 1000 in the posterior-anterior directions. The anterior flange 1112 can have a length that extends about 25% of the total thickness of the device 1000 in the posterior-anterior directions. The anterior flange 1112 can have a length that extends about 30% of the total thickness of the device 1000 in the posterior-anterior directions. The anterior flange 1112 can have a length that extends about 35% of the total thickness of the device 1000 in the posterior-anterior directions. The anterior flange 1112 can have a length that extends about 40% of the total thickness of the device 1000 in the posterior-anterior directions. The anterior flange 1112 can have a length that extends about 45% of the total thickness of the device 1000 in the posterior-anterior directions. The anterior flange 1112 can have a length that extends about 50% of the total thickness of the device 1000 in the posterior-anterior directions. The anterior flange 1112 can have a length that extends about 55% of the total thickness of the device 1000 in the posterior-anterior directions. The anterior flange 1112 can have a length that extends about 60% of the total thickness of the device 1000 in the posterior-anterior directions. The anterior flange 1112 can have a length that extends about 65% of the total thickness of the device 1000 in the posterior-anterior directions. The anterior flange 1112 can have a length that extends about 70% of the total thickness of the device 1000 in the posterior-anterior directions. The anterior flange 1112 can have a length that extends about 75% of the total thickness of the device 1000 in the posterior-anterior directions. The anterior flange 1112 can have a length that extends about 80% of the total thickness of the device 1000 in the posterior-anterior directions. The anterior flange 1112 can have a length that extends about 85% of the total thickness of the device 1000 in the posterior-anterior directions. The anterior flange 1112 can have a length that extends about 90% of the total thickness of the device 1000 in the posterior-anterior directions.

In some embodiments, the posterior flange 1110 can extend around the entire perimeter of the posterior interior surface 1106. In some embodiments, the posterior flange 1110, can extend around a portion of the perimeter of the posterior interior surface 1106. In some embodiments, the posterior flange 1110 can comprise one or more flange segments disposed around the perimeter of the posterior interior surface 1106. In some embodiments, the anterior flange 1112 can extend around the entire perimeter of the anterior interior surface 1108. In some embodiments, the anterior flange 1112, can extend around a portion of the perimeter of the anterior interior surface 1108. In some embodiments, the anterior flange 1112 can comprise one or more flange segments disposed around the perimeter of the anterior interior surface 1108.

In some embodiments, the posterior flange 1110 can be configured to retain one or more intraocular lenses, electronic devices, or other intraocular devices within the cavity 1114. In some embodiments, the anterior flange 1112 can be configured to retain one or more intraocular lenses, electronic devices, or other intraocular devices within the cavity 1114.

The posterior flange 1110 can be configured to rigidly resist deflection. The posterior flange 1110 can be configured to permissively resist deflection. The anterior flange 1112 can be configured to rigidly resist deflection. The anterior flange 1112 can be configured to permissively resist deflection.

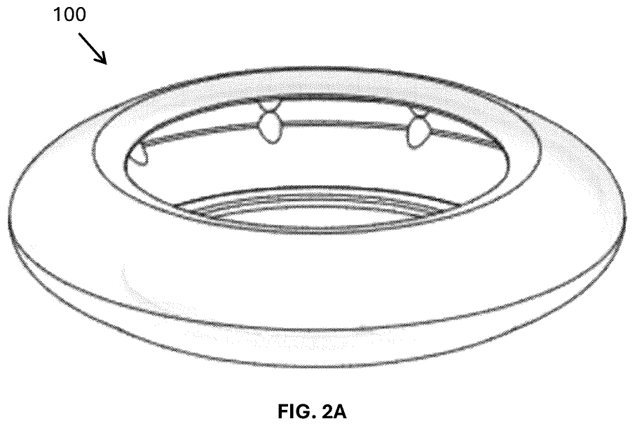

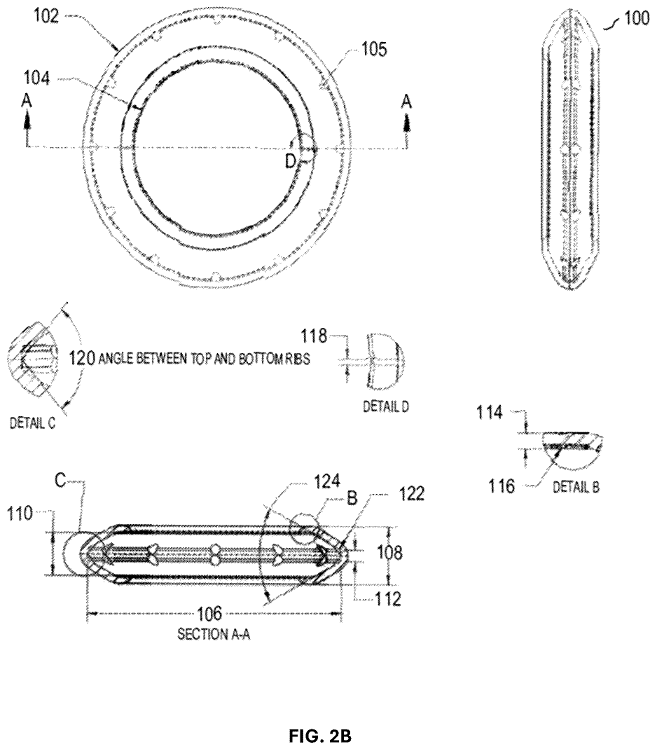

FIGS. 2A-2B illustrates various perspective views of an example of a prosthetic capsular device 100. In some embodiments, the device 100 includes features described with respect to the devices described in U.S. Pat. No. 10,603,162, which is hereby incorporated by reference in its entirety, or modifications thereof. The prosthetic device 100 may comprise any prosthetic device as described herein with reference to FIGS. 1A-1B.

In some embodiments, the device 100 includes features described with respect to the devices described in U.S. Pat. No. 9,358,103, which is hereby incorporated by reference in its entirety, or modifications thereof. For example, the device 100 can comprise an anterior side, a posterior side, and one or more sidewalls extending between the anterior side and the posterior side; a cavity or opening defined by the anterior side, posterior side, and the one or more sidewalls. Device 100 can be configured to comprise one or more intraocular lenses, electronic devices, drug delivery devices, or other intraocular devices held within the cavity. The IOLs may comprise any and all lens powers and designs that are currently known in the art of intraocular lenses, including, but not limited to: spherical, aspheric, wavefront, convex, concave, extended depth of focus, pinhole or small aperture, multifocal (diffractive, refractive, zonal), toric, accommodative, ultraviolet (UV) filtering, diffractive chromatic aberration reducing lenses, light adjustable lenses (ultraviolet light adjustable, femtosecond phase wrapping), and optical powers ranging from any positive diopter value (e.g., including +35 D and above) to any negative diopter value (e.g., including −35 D and below).

Further, in certain embodiments, device 100 includes one or more additional features. For example, device 100 can comprise a generally lenticular or lens-like shape as opposed to a box-like design. In other words, the generally shape of the device 100 can be more like the shape of a natural lens. Risks of negative and/or positive dysphotopsia can be reduced due to the generally lenticular shape of the device 100. Negative dysphotopsia is a widespread problem in cataract surgery, generally described by patients as a temporal dark crescent in their vision and is believed to occur either due to the optical phenomenon known as total internal reflection or by obstruction of light. This can occur either at the junction of the optic edge and the empty collapsed surrounding capsule forming a relatively planar surface, or due to the capsule overlapping a portion of the optic, most commonly the nasal aspect. In embodiments in which the implantable device 100 comprises an overall lens-like configuration, the capsule can be held open, preventing a relatively planar surface from being formed by fusion of the posterior and anterior capsule. More specifically, when light hits a curvilinear slice of the device 100, which can be made from silicone for example, it may travel through the curvilinear slice instead of bouncing off and causing a negative shadow as it generally would for flat surfaces. This may be especially true in the horizontal meridian across the 180-degree plane. As such, in some embodiments, device 100 does not comprise any flat edges or surfaces. In other words, every surface of the device 100 can be curvilinear. Flat optical surfaces can promote total internal reflection and are not found in the natural human lens or lens capsule in the native state. One goal of some of the embodiments described herein is to reduce negative dysphotopsias by not having any flat optical surfaces. Certain embodiments may have additional features such as an opaque or translucent tint. This may further enhance the reduction of positive dysphotopsias by blocking stray light that could be reflected off of the IOL border or haptic edges. This could also function as an artificial iris of sorts, depending on the color and opacity of the tint, blocking light that could be transmitted through an iris transillumination defect, a traumatically altered iris, or a surgical peripheral iridotomy, likewise preventing positive dysphotopsias and glare.

In some embodiments, substantially the whole device 100 can comprise silicone and/or a soft silicone polymer or other materials as described throughout this application. In addition, in certain embodiments, substantially the whole device 100 can comprise a flexible and/or elastic material. As such, device 100 can be foldable or collapsible for implantation into the eye through a small incision. Once inserted into the eye, device 100 can naturally unfold and self-expand into its expanded configuration as illustrated in FIG. 1. Device 100 can comprise one or more capsular areas. The one or more capsular areas can be adapted to receive and/or hold an IOL and/or one or more drug delivery devices. In some embodiments, one or more sidewalls can comprise a concave shape. For example, the interior surface of the one or more sidewalls can form a cavity. The cavity can be configured to hold an IOL or drug delivery device, for example.

In some embodiments, device 100 comprises a single-molded design. In other words, the whole device 100, or substantially the whole device 100 can be molded from a single piece of material. For example, in some embodiments, substantially the whole device 100 can be molded of silicone using a silicone compression mold. In other embodiments, the device 100 or any portion thereof can be manufactured by 3D laser cutting, two photon lithography, additive manufacturing, 3D printing, compression molding, and/or any combination of the aforementioned manufacturing processes or others.

In some embodiments, the device 100 can be inserted through an incision between about 1.5 mm and about 3 mm (e.g., about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, ranges between such values, etc.).

Further, in some embodiments, a length of a major axis of the device 100 or a length measured from the outermost end of one sidewall to the outermost end of another sidewall along a major axis of the device 100 can be about 9.65 mm. In other embodiments, the length of the major axis of the device 100 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 100 may comprise a diameter 102 of the device 100.

In some embodiments, the thickness of silicone or other material of the device 100 can be about 0.25 mm. In certain embodiments, the thickness of silicone or other material of the device 100 can be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or within a range defined by two of the aforementioned values.

In some embodiments, the thickness of the silicone or other material of the device 100 varies depending on the portion of the device 100. In other words, some portions of device 100 can be made of thinner materials while other portions of device 100 can be made of thicker materials. For example, certain portions of the device that provide support to the anterior portion of the device 100 may be made with thicker materials for added support.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 104, which can be a diameter of an anterior opening and/or a posterior opening.

In some embodiments, the shape and size of the device 100 may minimize anterior, posterior, and/or radial protrusion into the natural capsular bag relative to previously used capsular devices. In some embodiments, the device 100 may be smaller in certain dimensions especially towards the anterior and periphery of the device. In some embodiments, sharp and/or straight edges or sides may not be present in the device 100 to reduce friction between the device 100 and the posterior aspect of the iris of an eye. In some embodiments, the smaller size, decrease in anterior, posterior, and/or radial protrusion into the natural capsular bag, and smoothened or curved edges, may result in the device 100 having an enhanced biocompatibility profile and/or may reduce inflammation caused by the device in the eye. In some embodiments, the unique shape and design of device 100 may result in a decrease and/or elimination of inflammation of the eye (e.g., anterior of the eye) upon insertion of the device. In some embodiments, the decrease and/or elimination of post-insertion inflammation resulting from the shape of the device 100 may result in a decrease and/or elimination of the need for post-operative anti-inflammatory medications such as, e.g., steroids or nonsteroidal anti-inflammatory drugs (NSAIDs). It may also result in a decrease and/or elimination of device removals and/or replacements, which may be needed if inflammation cannot be reduced or removed.

The device 100 can be self-expandable to keep the capsule open. The device 100 can comprise at least three different planes. For example, a first plane can correspond with the posterior opening or end of the device, where an IOL can be attached. A second plane can correspond with the anterior opening or end of the device, where another refractive surface or IOL can be attached. In some embodiments, device 100 comprises a symmetrical device such that the anterior opening and posterior opening are determined by the device 100 position in the eye. A third plane can be positioned in between the posterior end and the anterior end, for example, along a ridge formed in the central portion, where another an IOL can be attached. In some embodiments, the central portion may comprise a continuous lateral portion interposed between the anterior portion and the posterior portion. In some embodiments, the continuous lateral portion protrudes radially beyond the anterior portion and the posterior portion. In some embodiments, the continuous lateral portion fully encloses a lateral side of the housing structure, wherein an internal cavity of the continuous lateral portion forms a groove for containing an IOL. In some embodiments, the central portion may comprise a continuous lateral portion comprising an exterior surface comprising a rounded bulge, the rounded bulge extending radially beyond the anterior portion and the posterior portion. In some embodiments, the continuous lateral portion comprises an interior surface comprising a groove or ridge, wherein at least a portion of the interior surface is formed at an acute angle or an obtuse angle relative to the anterior portion and the posterior portion.

In some embodiments, the prosthetic capsular devices comprise one or more orientation designation indicators or mechanisms 118 configured to serve as a marker to indicate the direction and/or orientation of the prosthetic device before, during, and/or after insertion into the eye. In some embodiments, the one or more orientation designation mechanisms 118 may be located on the anterior side, the posterior side, and/or on the interior and/or exterior sidewalls of the prosthetic capsular device. In some embodiments, the one or more orientation designation mechanisms 118 may assist and/or allow a surgeon or medical professional to determine or perceive if the prosthetic capsular device is oriented correctly before, during, and/or after insertion into the eye.

In some embodiments, the one or more orientation indicators may comprise visual distinguishing factors on the anterior side, the posterior side, and/or on the interior and/or exterior sidewalls of the prosthetic capsular device. For example, the anterior side, the posterior side, and/or the interior and/or exterior sidewalls may differ based on varying structural features, axis marks, colors, shapes, textures, tones, shades, brightness, outlines, sizes, text indicators, engravings, and icons, among others. In some embodiments, the one or more orientation designation indicators facilitate the current orientation of the prosthetic capsular device before, during, and after insertion into the eye and serve as measurement tools to measure, for example, rotational stability.

In some embodiments, the one or more orientation designation indicators comprise a protuberance, nub, protrusion, projection, bulge, or other structure extending from a surface of the housing 100. In some embodiments, the one or more orientation designation indicators comprise a visual marker such as a hole or aperture. In some embodiments, the visual marker may serve as a reference point to measure rotational stability and position of the prosthetic capsular device 100 before, during, and/or after insertion into the eye. In some embodiments, the one or more orientation designation indicators 118 may extend radially inward from the diameter 104 of the anterior opening and/or the posterior opening. However, in some embodiments, the one or more orientation designation indicators may extend radially inward or radially outward from any structure of the prosthetic device 100 and/or an IOL coupled to the device. In some embodiments, it may be preferable for the one or more orientation designation indicators to extend radially inwardly from the anterior opening to provide optimal visibility to a surgeon and/or medical professional and to avoid unnatural exterior protrusions into the natural capsular bag.

In some embodiments, the prosthetic capsular device 100 may comprise about 2 orientation designation indicators. In some embodiments, the number of orientation designation indicators 118 may be about 1, 2, 3, 4, 5, 6, 7, 8, 9, 10, 11, 12, 13, 14, 15, 20, 25, 30, 35, 40, 45, 50, 100, and/or within a range defined by two of the aforementioned values. An example orientation designation indicator 118 is illustrated in Detail D of FIG. 2B. In some embodiments, the orientation designation indicator 118 may comprise a thickness of about 0.10 mm. In some embodiments the orientation designation indicator 118 may comprise a thickness of between about 0.01 mm and 0.30 mm.

In some embodiments, a prosthetic capsular device configured to be inserted in a natural capsular bag of an eye after removal of a lens can comprise a housing structure 100 capable of containing one or more intraocular devices and/or refractive surfaces. In particular, the housing structure can comprise an anterior side, wherein the anterior side comprises an anterior opening that can be elliptical, circular, arcuate, triangular, rectangular, polygonal, or otherwise shaped as shown in the provided Figures, wherein the anterior opening is capable of allowing at least one of insertion, removal, or replacement of an intraocular lens device, and wherein the anterior opening is further configured to be coupled to a lens to cover the anterior opening; a posterior side, wherein the posterior side comprises an posterior opening that can be elliptical, circular, arcuate, triangular, rectangular, polygonal, or otherwise shaped as shown in the provided Figures, wherein the posterior opening is capable of allowing at least one of insertion, removal, or replacement of an intraocular lens or device, and wherein the posterior opening is further configured to be coupled to an intraocular lens or device to cover the posterior opening; and a continuous lateral portion interposed between the anterior portion and the posterior portion, wherein the continuous lateral portion protrudes radially beyond the anterior portion and the posterior portion, wherein the continuous lateral portion fully encloses a lateral side of the housing structure, wherein an internal cavity of the continuous lateral portion forms a groove or ridge for containing an intraocular lens or device within, for example, an anterior portion of the device. The continuous lateral portion may not have any openings, for example along the lateral portion of the device in some embodiments. The housing structure 100 can be symmetrical over a plane at a midpoint of the continuous lateral portion between the anterior portion and the posterior portion.

In some embodiments, the ridge or groove may comprise one or more ribs 105. The ribs 105 are shown in detail along the ridge as Detail C in FIG. 2B. In some embodiments, the ribs may be configured to hold an intraocular lens within device 100. For example, the ribs 105 may be configured to reduce mobility of an intraocular lens within device 100, such that lens tilt, lens rotation, and/or decentration is reduced or eliminated. In some embodiments, the ribs 105 may comprise a top surface and a bottom surface with a rib angle 120 between the top and bottom surface. In some embodiments, the rib angle may comprise about 100°. In some embodiments, the rib angle may comprise about 10° to about 180°. For example, in some embodiments, the rib angle may be about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, about 175°, about 180°, or any value between the aforementioned values. In some embodiments, the rib angle 105 may be determined based on the thickness 108 of the device 100, along with the diameter 102.

In some embodiments, device 100 may comprise about 12 ribs. In some embodiments, the device 100 may comprise between about 1 rib and 100 ribs. about 1 ribs, about 2 ribs, about 3 ribs, about 4 ribs, about 5 ribs, about 6 ribs, about 7 ribs, about 8 ribs, about 9 ribs, about 10 ribs, about 11 ribs, about 12 ribs, about 13 ribs, about 14 ribs, about 15 ribs, about 16 ribs, about 17 ribs, about 18 ribs, about 19 ribs, about 20 ribs, about 21 ribs, about 22 ribs, about 23 ribs, about 24 ribs, about 25 ribs, about 26 ribs, about 27 ribs, about 28 ribs, about 29 ribs, about 30 ribs, about 31 ribs, about 32 ribs, about 33 ribs, about 34 ribs, about 35 ribs, about 36 ribs, about 37 ribs, about 38 ribs, about 39 ribs, about 40 ribs, about 41 ribs, about 42 ribs, about 43 ribs, about 44 ribs, about 45 ribs, about 46 ribs, about 47 ribs, about 48 ribs, about 49 ribs, about 50 ribs, about 51 ribs, about 52 ribs, about 53 ribs, about 54 ribs, about 55 ribs, about 56 ribs, about 57 ribs, about 58 ribs, about 59 ribs, about 60 ribs, about 61 ribs, about 62 ribs, about 63 ribs, about 64 ribs, about 65 ribs, about 66 ribs, about 67 ribs, about 68 ribs, about 69 ribs, about 70 ribs, about 71 ribs, about 72 ribs, about 73 ribs, about 74 ribs, about 75 ribs, about 76 ribs, about 77 ribs, about 78 ribs, about 79 ribs, about 80 ribs, about 81 ribs, about 82 ribs, about 83 ribs, about 84 ribs, about 85 ribs, about 86 ribs, about 87 ribs, about 88 ribs, about 89 ribs, about 90 ribs, about 91 ribs, about 92 ribs, about 93 ribs, about 94 ribs, about 95 ribs, about 96 ribs, about 97 ribs, about 98 ribs, about 99 ribs, or about 100 ribs.

In some embodiments, a thickness 108 of the device may comprise a maximum distance between the anterior side and posterior side of the device 100. In some embodiments, the thickness 108 of device 100 may be about 2.00 mm. In some embodiments, the thickness 108 of the device 100 may be about 1.50 mm. In some embodiments, the thickness 108 of the device 100 may be between about 0.5 mm and 4.0 mm. In some embodiments, the thickness 108 of the device 100 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, device 100 may comprise a ridge thickness 112 comprising the size of the ridge locating ribs 105. In some embodiments, the ridge thickness 112 may be about 0.40 mm. In some embodiments, the ridge thickness 112 may be between about 0.10 mm and about 1.00 mm. In some embodiments, the ridge thickness may be configured to reduce the possibility of lens tilt by an intraocular lens located in the ridge and secured by ribs 105.

In some embodiments, device 100 may comprise an inner thickness 110 comprising a distance between an inner surface of the sidewall at the anterior opening and an inner surface of the sidewall at the posterior opening. In some embodiments, the inner thickness 110 may be about 1.48 mm.

In some embodiments, device 100 may comprise an inner diameter 106 comprising the distance between the interior surfaces of the sidewalls at the ridge. In some embodiments, the inner diameter 106 may be about 9.15 mm. In some embodiments, the interior diameter may be between about 5.00 mm and 15.00 mm.

In some embodiments, the sidewall at the anterior portion of the device 100 and the sidewall at the posterior of the device 100 may form a sidewall angle 124, formed at the ridge of the device 100. In some embodiments, the sidewall angle 124 may be about 34°. In some embodiments, the sidewall angle 124 may about 57°. In some embodiments, the sidewall angle 124 may be about 10° to about 180°. For example, in some embodiments, the sidewall angle 124 may be about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, about 175°, about 180°, or any value between the aforementioned values. In some embodiments, the sidewall angle 124 may be determined based on the thickness 108 of the device 100, along with the diameter 102.

In some embodiments, the cavity of the device may comprise a volume for maintaining the shape and size of the natural capsular bag. The volume may be formed by the angled sidewalls and may comprise a tapered confinement area, wherein the sidewall taper into the cavity at the anterior and posterior openings. In some embodiments, the taper may form a curve in the sidewall adjacent to the anterior and posterior openings. The curved section of the sidewall may provide a smoothed edge to reduce the impact of incidental contact with the posterior surface of the iris of the eye, or pressure transduced through the natural capsular bag and imparted onto the posterior surface of the iris of the eye. In some embodiments, the taper length 114 may comprise a distance between the exterior surface of the sidewall at its most anterior/posterior point and the exterior surface at the anterior/posterior opening. In some embodiments, the exterior surface may comprise curved surfaces 122 and 116 at the ridge and at the openings, respectively. The curved shape of the sidewalls may contribute to a reduction in post-surgical complications through minimization of contact or the severity of contact between the device 100 and the iris. The curved shape of the sidewall of the device 100 near the openings is shown in Detail B of FIG. 2B.

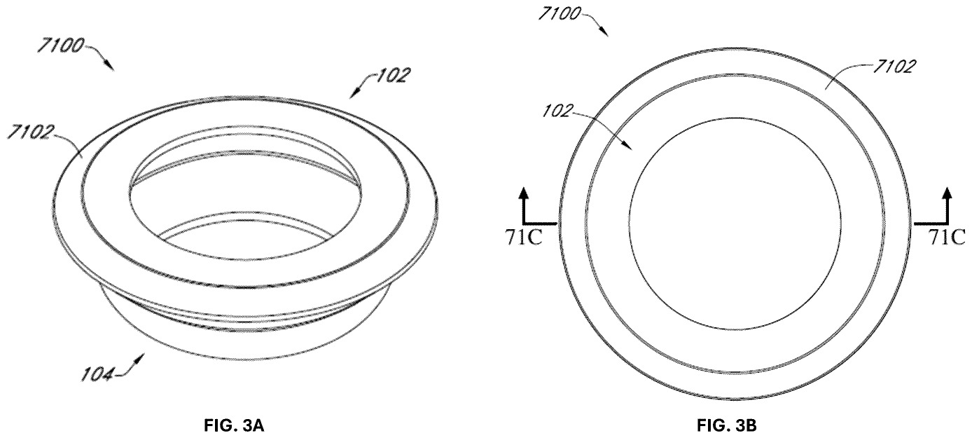

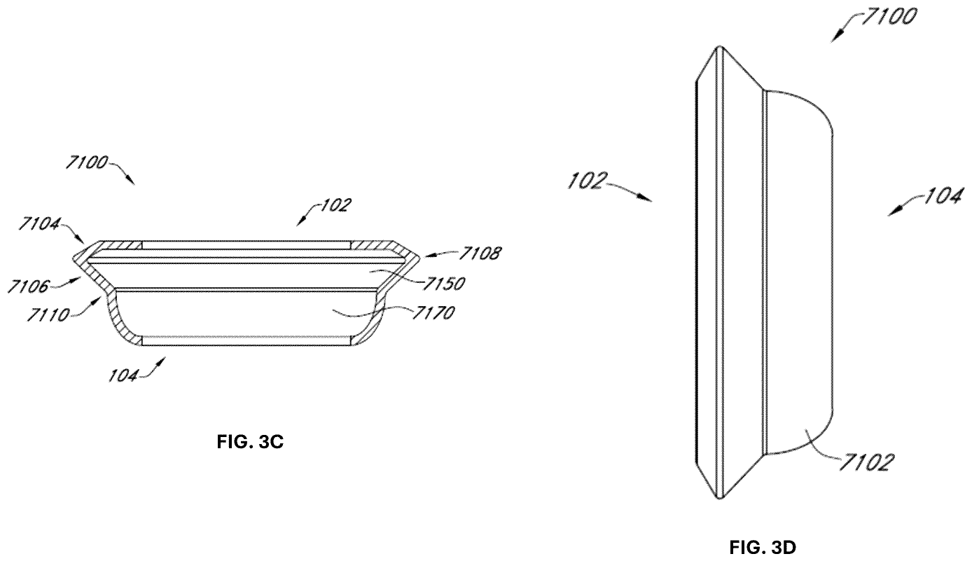

FIG. 3A is an anterior side perspective view of another example prosthetic capsular device. FIG. 3B is an anterior plan view of the example prosthetic capsular device of FIG. 3A. FIG. 3C is a cross-sectional view of the example prosthetic capsular device of FIG. 3A along the line A-A of FIG. 3B. FIG. 3D is a side plan view of the example prosthetic capsular device of FIG. 3A.

The example prosthetic capsular device 7100 illustrated in FIG. 3A-3D can include a single continuous sidewall 7102, a posterior opening or end 104, an anterior opening or end 102, an anterior portion 7150 and a posterior portion 7170. In some embodiments, the example prosthetic capsular device 7100 can comprise dimensions smaller than, similar to, or identical to those described in relation to the example prosthetic capsular devices of FIGS. 1A-2B and/or the other prosthetic capsular devices described herein, and the dimensions can be tailored to a particular eye being operated on. For example, the device 7100 can include the flanges 1110, 1112 of FIGS. 1A-1B.

In some embodiments, certain symmetrical capsular devices may incite inflammation in a patient's eye due to iris chaffing and/or irritation from pressure that the capsular devices may place on the posterior iris. Because of the symmetrical nature of the design, inflammation of the eye may result. The human lens and/or natural capsular bag may have an asymmetrical shape such that it is flatter on the anterior surface and more concave on the posterior surface. In some embodiments, the prosthetic capsular devices of FIGS. 3A-3D may comprise a design, such as an asymmetric shape comprising a flatter anterior portion and a more concave or steeper posterior portion for example, that prevents anterior translation of pressure, and thus prevents iris chaffing or contact and resulting inflammation of the eye of a patient. In some embodiments, the prosthetic capsular devices may be slightly, partly, substantially, or highly asymmetric. In some embodiments, the asymmetric nature of the prosthetic capsular devices may require the devices to be loaded and inserted into the eye in a very specific manner, such that the device is oriented correctly within the eye, for example in a preferred anterior-posterior orientation. In some embodiments, the prosthetic capsular devices comprise one or more orientation designation indicators or mechanisms configured to serve as a marker to indicate the direction and/or orientation of the prosthetic device before, during, and/or after insertion into the eye. In some embodiments, the one or more orientation designation mechanisms may be located on the anterior side, the posterior side, and/or on the interior and/or exterior sidewalls of the prosthetic capsular device. In some embodiments, the one or more orientation designation mechanism may assist and/or allow a surgeon or medical professional to determine or perceive if the prosthetic capsular device is oriented correctly before, during, and/or after insertion into the eye.

In some embodiments, the one or more orientation indicators may comprise visual distinguishing factors on the anterior side, the posterior side, and/or on the interior and/or exterior sidewalls of the prosthetic capsular device. For example, the anterior side, the posterior side, and/or the interior and/or exterior sidewalls may differ based on varying structural features, axis marks, colors, shapes, textures, tones, shades, brightness, outlines, sizes, text indicators, engravings, and icons, among others. In some embodiments, the one or more orientation designation indicators facilitate the current orientation of the prosthetic capsular device before, during, and after insertion into the eye and serve as measurement tools to measure, for example, rotational stability.

In some embodiments, the shape and size of the device 7100 may minimize anterior, posterior, and/or radial protrusion into the natural capsular bag. In some embodiments, the device 7100 may be smaller in certain dimensions especially towards the anterior and/or periphery of the device. In some embodiments, the smaller size and/or decrease in anterior, posterior, and/or radial protrusion into the natural capsular bag may result in the device 7100 having an enhanced biocompatibility profile. In some embodiments, the unique shape of the design of the device 7100 may result in a decrease and/or elimination of inflammation of the eye (e.g. anterior of the eye) upon insertion of the device. In some embodiments, the decrease and/or elimination of post-insertion inflammation resulting from the shape of the device 7100 may result in a decrease and/or elimination of the need for post-operative anti-inflammatory medications such as, e.g., steroids or nonsteroidal high inflammatory medications. It may also result in a decrease and/or elimination of device removals and/or replacements, which be needed if inflammation cannot be reduced or removed.

In some embodiments, the device 7100 can be self-expandable to keep the capsule fully open. The device 7100 can comprise at least three different planes, which can be substantially perpendicular to an anterior-posterior or longitudinal axis of the device in some embodiments. For example, a first plane can correspond with the posterior opening or end 104 of the device, where a refractive surface or IOL can be attached. A second plane can correspond with the anterior opening or end 102 of the device, where another refractive surface or IOL can be attached. A third plane can be positioned in between the posterior end and the anterior end, for example, along a ridge formed in the anterior portion 7150, as shown in FIG. 3C, where another refractive surface, technology device, drug delivery device, IOL, and/or any other intraocular device can be attached.

In some embodiments, the anterior opening or end 102 may comprise an opening and radially outward flat surface. In some embodiments, the continuous sidewalls 7102 may extend angularly and radially outwards from the anterior end 102, as shown in FIG. 3C. In some embodiments, the continuous sidewalls 7102 may comprise one or more straight walls in the anterior portion 7150 of the prosthetic capsular device. In some embodiments, the continuous sidewalls 7102 may comprise two sections within the anterior portion 7150. In some embodiments, the continuous sidewalls 7102 may comprise a radially outward 7104 portion adjacent to the anterior end 102 and a radially inward portion 7106 bridged to the radially outward portion 7104 by an anterior transition point 7108. In some embodiments, the radially outward portion 7104 and the radially inward portion 7106 may be separated at an angle of substantially 90°. In some embodiments, the radially outward portion 7104 and the radially inward portion 7106 may be separated at an angle of about 10°, about 20°, about 30°, about 40°, about 50°, about 60°, about 70°, about 80°, about 90°, about 100°, about 110°, about 120°, about 130°, about 140°, about 150°, about 160°, about 170°, about 180°, and/or within a range defined by two of the aforementioned values.

In some embodiments, the radially outward portion 7104 of the continuous sidewalls 7102 may form a trapezoidal shape when viewed from the side and/or in a cross-sectional view, such as in FIG. 3C. In some embodiments, the anterior transition point 7108 of the continuous sidewalls 7102 may comprise the base of the trapezoid and the anterior end or opening 102 may comprise the upper edge of the trapezoid. In some embodiments, the trapezoidal shape forms a three dimensional circular or arcuate truncated cone structure in the anterior portion 7150.

In some embodiments, the anterior transition point 7108 may comprise a circular or otherwise arcuate rounded edge of the prosthetic capsular device. In some embodiments, the anterior transition point 7108 may be located on a radially outermost diameter of the device 7100. In some embodiments, the outermost diameter of the device 7100 may be about 10.00 mm. In some embodiments, the outermost diameter of the device 7100 may be about 3.00 mm, about 4.00 mm, about 5.00 mm, about 5.50 mm, about 6.00 mm, about 6.50 mm, about 7.00 mm, about 7.50 mm, about 8.00 mm, about 8.50 mm, about 9.00 mm, about 9.50 mm, about 10.00 mm, about 10.50 mm, about 11.00 mm, about 11.50 mm, about 12.00 mm, about 12.50 mm, about 13.00 mm, about 14.00 mm, about 15.00 mm, and/or within a range defined by two of the aforementioned values.

In some embodiments, the anterior transition point 7108 may comprise a rounded edge having a radius of about 0.1 mm. In some embodiments, the transition point 7108 may comprise a rounded edge having a radius of about 0.01 mm, about 0.02 mm, about 0.03 mm, about 0.04 mm, about 0.05 mm, about 0.06 mm, about 0.07 mm, about 0.08 mm, about 0.09 mm, about 0.10 mm, about 0.11 mm, about 0.12 mm, about 0.13 mm, about 0.14 mm, about 0.15 mm, about 0.16 mm, about 0.17 mm, about 0.18 mm, about 0.19 mm, about 0.20 mm, about 0.25 mm, about 0.30 mm, about 0.40 mm, about 0.50 mm, about 0.60 mm, about 0.70 mm, about 0.80 mm, about 0.90 mm, about 1.00 mm and/or within a range defined by two of the aforementioned values.

In some embodiments, the prosthetic capsular device 7100 comprises a thickness or width, the thickness ranging from the posterior opening or end 104 to the anterior opening or end 102. In some embodiments, the thickness of the capsular device 7100 is about 3.00 mm. In some embodiments the thickness of the device 7100 can be about 0.50 mm, about 1.00 mm, about 1.50 mm, about 2.00 mm, about 2.50 mm, about 3.00 mm, about 3.50 mm, about 4.00 mm, about 4.50 mm, about 5.00 mm, about 5.50 mm, about 6.00 mm, about 6.50 mm, about 7.00 mm, about 8.00 mm, about 9.00 mm, about 10.00 mm, and/or within a range defined by two of the aforementioned values.

In some embodiments, anterior transition point 7108 can be located about 2.50 mm, along a longitudinal axis of the device, from the posterior end or opening 104. Along a longitudinal axis of the device, the anterior transition point 7108 may be located about 0.20 mm, about 0.25 mm, about 0.30 mm, about 0.40 mm, about 0.50 mm, about 0.60 mm, about 0.70 mm, about 0.80 mm, about 0.90 mm, about 1.00 mm, about 1.50 mm, about 2.00 mm, about 2.50 mm, about 3.00 mm, about 3.50 mm, about 4.00 mm, about 4.50 mm, about 5.00 mm, about 5.50 mm, about 6.00 mm, about 6.50 mm, about 7.00 mm, about 8.00 mm, about 9.00 mm, about 10.00 mm, and/or within a range defined by two of the aforementioned values from the posterior end or opening 104.

In some embodiments, the anterior end or opening 102 may comprise a diameter of about 6.00 mm. In some embodiments, the anterior end or opening 102 may comprise a diameter of about 3.00 mm, about 4.00 mm, about 5.00 mm, about 5.50 mm, about 6.00 mm, about 6.50 mm, about 7.00 mm, about 7.50 mm, about 8.00 mm, about 8.50 mm, about 9.00 mm, about 9.50 mm, about 10.00 mm, about 10.50 mm, about 11.00 mm, about 11.50 mm, about 12.00 mm, about 12.50 mm, about 13.00 mm, about 14.00 mm, about 15.00 mm, and/or within a range defined by two of the aforementioned values.

In some embodiments, the posterior end or opening 102 may comprise a diameter of about 6.00 mm. In some embodiments, the posterior end or opening 102 may comprise a diameter of about 3.00 mm, about 4.00 mm, about 5.00 mm, about 5.50 mm, about 6.00 mm, about 6.50 mm, about 7.00 mm, about 7.50 mm, about 8.00 mm, about 8.50 mm, about 9.00 mm, about 9.50 mm, about 10.00 mm, about 10.50 mm, about 11.00 mm, about 11.50 mm, about 12.00 mm, about 12.50 mm, about 13.00 mm, about 14.00 mm, about 15.00 mm, and/or within a range defined by two of the aforementioned values.

In some embodiments, a central transition point 7110 may link the anterior portion 7150 and the posterior portion 7170 of the prosthetic device 7100. The central transition point 7110 may comprise a point at which the continuous sidewalls 7102 transform from a straight wall on the, for example, radially inward section 7106 of the anterior portion 7150 to a curvilinear sidewall on the posterior portion 7170. The curvilinear sidewall on the posterior portion 7170 may taper inwardly towards the posterior end or opening 104.

In some embodiments, the radially inward portion 7106 of the continuous sidewalls 7102 may form an inverted trapezoidal shape when viewed from the side and/or cross-sectional view, such as in FIG. 3C. In some embodiments, the anterior transition point 7108 of the continuous sidewalls 7102 may comprise the upper or top edge of the inverted trapezoid and the central transition point 7110 may comprise the base of the inverted trapezoid. In some embodiments, the inverted trapezoidal shape forms a three dimensional circular or arcuate inverted truncated cone structure in the anterior portion 7150. In some embodiments, the inverted truncated cone structure mirrors the truncated cone structure formed by the radially outward section 7104, as described above. In some embodiments, the inverted truncated cone structure and the truncated cone structure comprise identical sizes and shapes. However, as shown in FIG. 3C, in some embodiments, the inverted truncated cone structure and the truncated cone structure may comprise different sizes and shapes depending on the location of the anterior transition point 7108, the central transition point 7110, and the anterior end or opening 102 along a longitudinal axis of the device 7100.

In some embodiments, the inverted truncated cone structure may comprise a first interior volume or cavity within the anterior portion 7150. In some embodiments, the truncated cone structure described above may comprise a second interior volume or cavity within the anterior portion 7150. In some embodiments, the first volume may comprise about an equal volume as the second volume. In some embodiments, the first volume may comprise a volume of about 1/10, about ⅛, about ⅙, about ⅕, about ¼, about ⅓, about ½, about ⅔, about ¾, about ⅚, about ⅞, about 1, and/or within a range defined by two of the aforementioned values, the second volume. In some embodiments, the first volume may comprise a volume about 1.1, about 1.2, about 1.3, about 1.4, about 1.5, about 1.6, about 1.7, about 1.9, about 2, about 3, about 4, about 5, about 6, about 7, about 8, about 9, or about 10, and/or within a range defined by two of the aforementioned values, times as large as the second volume.

In some embodiments, the central transition point 7110 may comprise a rounded edge having a radius of about 0.1 mm. In some embodiments, the central transition point 7110 may comprise a rounded edge having a radius of about 0.01 mm, about 0.02 mm, about 0.03 mm, about 0.04 mm, about 0.05 mm, about 0.06 mm, about 0.07 mm, about 0.08 mm, about 0.09 mm, about 0.10 mm, about 0.11 mm, about 0.12 mm, about 0.13 mm, about 0.14 mm, about 0.15 mm, about 0.16 mm, about 0.17 mm, about 0.18 mm, about 0.19 mm, about 0.20 mm, about 0.25 mm, about 0.30 mm, about 0.40 mm, about 0.50 mm, about 0.60 mm, about 0.70 mm, about 0.80 mm, about 0.90 mm, about 1.00 mm and/or within a range defined by two of the aforementioned values.

In some embodiments, the central transition point 7110 can be located about 1.50 mm, along a longitudinal axis of the device, from the posterior end or opening 104. Along a longitudinal axis of the device, the central transition point 7110 may be located about 0.20 mm, about 0.25 mm, about 0.30 mm, about 0.40 mm, about 0.50 mm, about 0.60 mm, about 0.70 mm, about 0.80 mm, about 0.90 mm, about 1.00 mm, about 1.50 mm, about 2.00 mm, about 2.50 mm, about 3.00 mm, about 3.50 mm, about 4.00 mm, about 4.50 mm, about 5.00 mm, about 5.50 mm, about 6.00 mm, about 6.50 mm, about 7.00 mm, about 8.00 mm, about 9.00 mm, about 10.00 mm, and/or within a range defined by two of the aforementioned values from the posterior end or opening 104.

In some embodiments, a prosthetic capsular device configured to be inserted in a natural capsular bag of an eye after removal of a lens can comprise a housing structure 7100 capable of containing one or more intraocular devices and/or refractive surfaces. In particular, the housing structure can comprise an anterior side, wherein the anterior side comprises an anterior opening that can be elliptical, circular, arcuate, triangular, rectangular, and/or polygonal, wherein the anterior opening is capable of allowing at least one of insertion, removal, or replacement of the intraocular device, and wherein the anterior opening is further configured to be coupled to a refractive surface to cover the anterior opening; a posterior side, wherein the posterior side comprises an posterior opening that can be elliptical, circular, arcuate, triangular, rectangular, and/or polygonal, wherein the posterior opening is capable of allowing at least one of insertion, removal, or replacement of an intraocular device, and wherein the posterior opening is further configured to be coupled to a refractive surface to cover the posterior opening; and a continuous lateral portion interposed between the anterior portion and the posterior portion, wherein the continuous lateral portion protrudes radially beyond the anterior portion and the posterior portion, wherein the continuous lateral portion fully encloses a lateral side of the housing structure, wherein an internal cavity of the continuous lateral portion forms a groove or ridge for containing an intraocular device within, for example, an anterior portion of the device. The continuous lateral portion may not have any openings, for example along the lateral portion of the device in some embodiments. The housing structure 7100 can be asymmetrical over a plane perpendicular to a longitudinal axis of the structure 7100 at a midpoint of the continuous lateral portion between the anterior portion and the posterior portion. In certain embodiments, the refractive surface can comprise a plurality of tabs or haptics for affixing the refractive surface to at least one of the arcuate anterior opening, the arcuate posterior opening, and/or the ridge or groove within the housing device, wherein the plurality of tabs or haptics protrude from the refractive surface.

As discussed above, one or more refractive surfaces, IOLs, lenses, optics, drug delivery devices, and/or other intraocular devices can be placed in the device 7100 at the posterior opening 104 and/or anterior opening 102. For example, a surgeon may initially insert a device with a posterior refractive surface into an eye of a patient. Depending on the outcome, the surgeon may have the option to reposition the lens originally inserted. For example, if the original lens was placed into the posterior opening and the patient had a hyperopic outcome, the surgeon could reposition the lens into the anterior opening thereby inducing a myopic shift in the refraction. Alternatively or in conjunction with lens repositioning, a surgeon could insert a secondary IOL into the middle section of the capsule, or into the anterior opening of the device 7100 to obtain better results. In other words, a secondary IOL can be placed on or in the anterior opening or in combination with other maneuvers for refractive fine tuning. Moreover, a biometric sensor and/or another IOL can be placed in the interior of the device 7100 as well, for example along the a ridge or groove within the interior of the device, the ridge or groove located, for example, along the interior wall of the device at the anterior transition point 7108.

In some embodiments, the device 7100 can be made in a number of different sizes or scales to accommodate for different patient biometry. For example, there can be a large, medium, and small sized device 7100 (or any other combination of sizes) to accommodate for patients with different sized cataracts and/or natural capsular bags. By providing a number of devices 7100 of varying sizes, surgeons can be able to select a particular device and/or optic for insertion in a particular patient.

The anterior portion 7150 and the posterior portion 7170 can be configured to hold a refractive surface, IOL, or another intraocular device. For example, a refractive surface and/or IOL can be configured to be placed in and/or over the anterior portion 7150 and/or posterior portion 7170. The anterior portion 7150 and/or posterior portion 7170 can be configured to hold an intraocular device(s) specifically designed for use with the device 7100, for example comprising one or more features that allow fixation of the intraocular device(s) at the posterior portion 7170 and/or anterior portion 7150. As such, as a non-limiting example, the device 7100 can allow implantation of one, two, three or more lenses to obtain an optimal refractive power and/or a refractive power that is desired.

In certain embodiments, the outer diameter of the anterior portion 7150 and/or posterior portion 7170, the inner diameter of the anterior portion 7150 and/or posterior portion 7170 within the device 7100, the opening(s) of the anterior portion 7150 and/or posterior portion 7170 can be about 3.00 mm, about 4.00 mm, about 5.00 mm, about 5.50 mm, about 6.00 mm, about 6.50 mm, about 7.00 mm, about 7.50 mm, about 8.00 mm, about 8.50 mm, about 9.00 mm, about 9.50 mm, about 10.00 mm, about 10.50 mm, about 11.00 mm, about 11.50 mm, about 12.00 mm, about 12.50 mm, about 13.00 mm, about 14.00 mm, about 15.00 mm, and/or within a range defined by two of the aforementioned values.

In some embodiments, the sidewalls 7102 may have a thickness of about 0.250 mm. In some embodiments, the sidewalls 7102 may comprise a thickness of about 0.05 mm, about 0.10 mm, about 0.15 mm, about 0.20 mm, about 0.25 mm, about 0.30 mm, about 0.35 mm, about 0.40 mm, about 0.45 mm, about 0.50 mm, about 0.60 mm, about 0.70 mm, about 0.80 mm, about 0.90 mm, about 1.00 mm, and/or within a range defined by two of the aforementioned values.

In some embodiments, the device 7100 may comprise one or more angled, straight, and/or curvilinear sidewalls adjacent to the anterior opening 102 and/or the posterior opening 104. The angled sidewalls may comprise the anterior portion 7150 and/or posterior portion 7150. The angled sidewalls may comprise one or more straight walls extending from the anterior opening and/or posterior opening to the straight sidewall of the central portion.

In some embodiments, the device 7100 illustrated in FIGS. 3A-3D may comprise one or more elliptical cutouts or wedges that span a vertical portion (for example when viewed from the view of FIG. 3D) from the anterior to the posterior. In some embodiments, the elliptical cutouts may facilitate form fitting of the device within the natural capsular bag. For example, in a patient with a large capsular bag, the elliptical cutouts may be fully open to expand the device to form-fit. In a smaller natural capsular bag, the elliptical cutouts may be compressed together to facilitate a compatible fit.

In some embodiments, when the prosthetic capsular devices described herein, for example in connection with FIGS. 3A-3D, are inserted into the eye, the natural capsular bag may adjust and/or contract in size and/or shape to form-fit around the device. In some embodiments, a capsulorhexis used to insert the devices described herein may be less than 6.00 mm such that the capsulorhexis may be smaller than the anterior opening and/or posterior opening of the device. In some embodiments, the devices described herein may be housed or may sit in a middle portion of the natural capsular bag. In some embodiments, the midpoint of the natural capsular bag may substantially correspond with the midpoint of the device in the eye along a longitudinal axis of the device. In some embodiments, the device 7100 of FIGS. 3A-3D may be inserted into the eye without any IOLs, refractive surfaces, or other intraocular devices inserted therein. In some embodiments, the IOLs, refractive surfaces, and/or other intraocular devices can be inserted into the device 7100 of FIGS. 3A-3D after the device has been inserted into the eye.

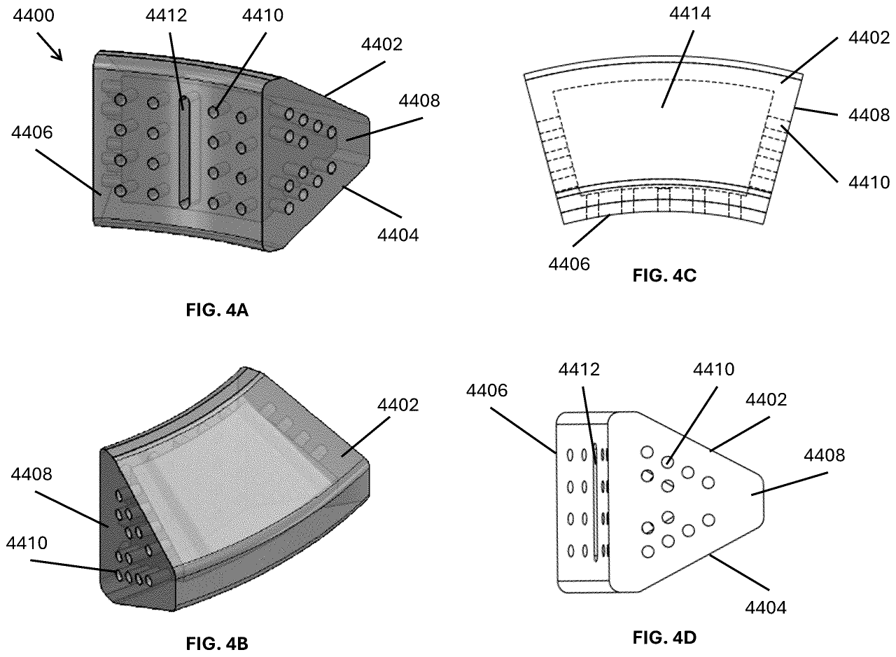

FIGS. 4A-4D illustrate an example drug delivery capsule 4400 according to some embodiments herein. FIGS. 4A and 4B illustrate first and second perspective views, FIG. 4C illustrates a cross-sectional view, and FIG. 4D is a side view.

In some embodiments, the capsule 4400 can comprise a structure having a posterior surface 4402 and an anterior surface 4404 which meet at an outer end of the capsule 4400. The capsule 4400 can comprise an interior surface 4406 having a shape of a section of a radial surface of a cylinder. The interior surface 4406 can be concave. The capsule 4400 can comprise a radial surface 4408 spanning from an inner end of the capsule 4400 to an outer end of the capsule 4400.

In some embodiments, the capsule 4400 can include diffusion openings 4410 (e.g. pores) disposed on one or more surface of the capsule 4400. The capsule 4400 can have openings 4410 disposed on the interior surface 4406. The capsule 4400 can have openings 4410 disposed on a radial surface 4408. The openings 4410 can span from an outer surface of the capsule 4400 to an inner cavity 4414 of the capsule 4400.

In some embodiments, the capsule 4400 can include a slit 4412 disposed on a surface of the capsule 4400. The capsule 4400 can include a slit 4412 disposed on the interior surface 4406 of the capsule 4400. The slit 4412 can span from an outer surface of the capsule 4400 to an inner cavity 4414 of the capsule 4400. In some embodiments, the cavity 4414 can be configured to receive a drug. The slit 4412 can be configured to receive an instrument (e.g. a syringe) for injecting a drug into the cavity 4414. In some embodiments, the cavity 4414 can be pre-filled with a drug during manufacture.

In some embodiments, the capsule 4400 can be configured to dispense a drug over time. A reserve of a drug can be stored in the cavity 4414. The drug can be dispensed through openings 4410. The openings 4410 can be configured to dispense a drug at a particular rate. The diameter of openings 4410 can be larger to promote more rapid dispensing of a drug. The diameter of openings 4410 can be smaller to promote a slower dispensing of a drug. The openings 4410 can comprise more openings to promote more rapid dispensing of a drug. The openings 4410 can comprise fewer openings to promote slower dispensing of a drug. In other embodiments, the size of the openings can be adjusted to control drug delivery.

In some embodiments, the capsule 4400 can be configured to dispense a solid drug. The solid drug can dissolve and elude through openings 4410. In some embodiments, the capsule 4400 can be configured to dispense a gel drug. The gel drug can dissolve and elude through openings 4410. In some embodiments, the capsule 4400 can be configured to dispense a polymerized drug. The polymerized drug can dissolve and elude through openings 4410. In some embodiments, the capsule 4400 can be configured to dispense a liquid drug. The liquid drug can elude through openings 4410.

In some embodiments, the capsule 4400 can be formed of a solid drug. The solid drug can dissolve and elude from the capsule 4400. In some embodiments, the capsule 4400 can be formed of a polymerized drug. The polymerized drug can dissolve and elude from the capsule 4400.

In some embodiments, the capsule 4400 can dispense more than one drug. The capsule 4400 can be formed of a first drug. The cavity 4414 can receive a second drug. The capsule 4400 can dispense a first drug as it dissolves and eludes from the capsule 4400. The capsule 4400 can dispense a second drug as it dissolves and eludes through openings 4410. The capsule 4400 can dispense a second drug as it eludes through openings 4410.

In some embodiments, the capsule 4400 can be symmetrical. In some embodiments, the capsule 4400 can be vertically symmetrical. In some embodiments, the capsule 4400 can be horizontally symmetrical. In some embodiments, the capsule 4400 can be radially symmetrical. In some embodiments, the capsule 4400 can be vertically symmetrical, horizontally symmetrical, and/or radially symmetrical.

In some embodiments, the capsule 4400 can be asymmetrical. In some embodiments, the capsule 4400 can be vertically asymmetrical. In some embodiments, the capsule 4400 can be horizontally asymmetrical. In some embodiments, the capsule 4400 can be radially asymmetrical. In some embodiments, the capsule 4400 can be vertically symmetrical, horizontally symmetrical, and/or radially asymmetrical.

In some embodiments, the capsule 4400 can have an angular width. The angular width can be about 20°. The angular width can be about 30°. The angular width can be about 360°, with the capsule 4400 forming a torus. The angular width can be between 20° and 360°. If the angular width is about 360°, the capsule 4400 can be continuous. If the angular width is about 360°, the capsule can include two radial surfaces disposed adjacent to each other.

In some embodiments, the capsule 4400 can be configured to fit within the cavity of the prosthetic device of FIGS. 1A-1B.

In some embodiments, the capsule 4400 can be configured to fit within the cavity of the prosthetic device of FIGS. 2A-2B. The exterior surface of the capsule 4400 can comprise features complementary to the features inside the cavity of the prosthetic device of FIGS. 2A-2B.

In some embodiments, the capsule 4400 can be configured to fit within the cavity of the prosthetic device of FIGS. 3A-3B. The capsule 400 can be shaped asymmetrically to match the features inside the cavity of the prosthetic device of FIGS. 3A-3B.

In some embodiments, more than one capsule 4400 can be configured to fit within the cavity of any of the prosthetic devices of FIGS. 1A-3B. Two capsules 4400 can be configured to fit within the cavity of any of the prosthetic devices of FIGS. 1A-3B. Three capsules 4400 can be configured to fit within the cavity of any of the prosthetic devices of FIGS. 1A-3B. Four capsules 4400 can be configured to fit within the cavity of any of the prosthetic devices of FIGS. 1A-3B. Five capsules 4400 can be configured to fit within the cavity of any of the prosthetic devices of FIGS. 1A-3B. More than five capsules 4400 can be configured to fit within the cavity of any of the prosthetic devices of FIGS. 1A-3B. The more than one capsules 4400 configured to fit within the cavity of any of the prosthetic devices of FIGS. 1A-3B can be configured to dispense the same drug or drugs. The more than one capsules 4400 configured to fit within the cavity of any of the prosthetic devices of FIGS. 1A-3B can be configured to dispense different drugs.

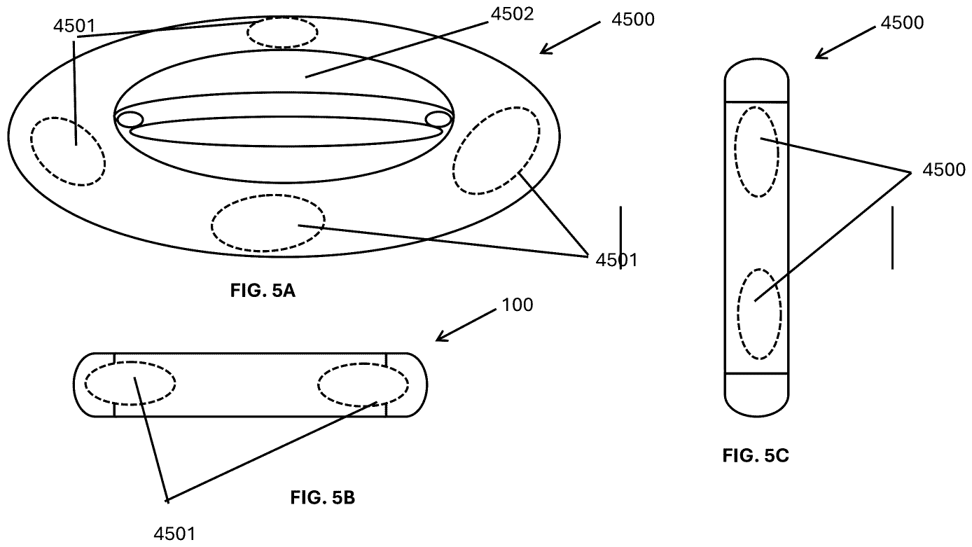

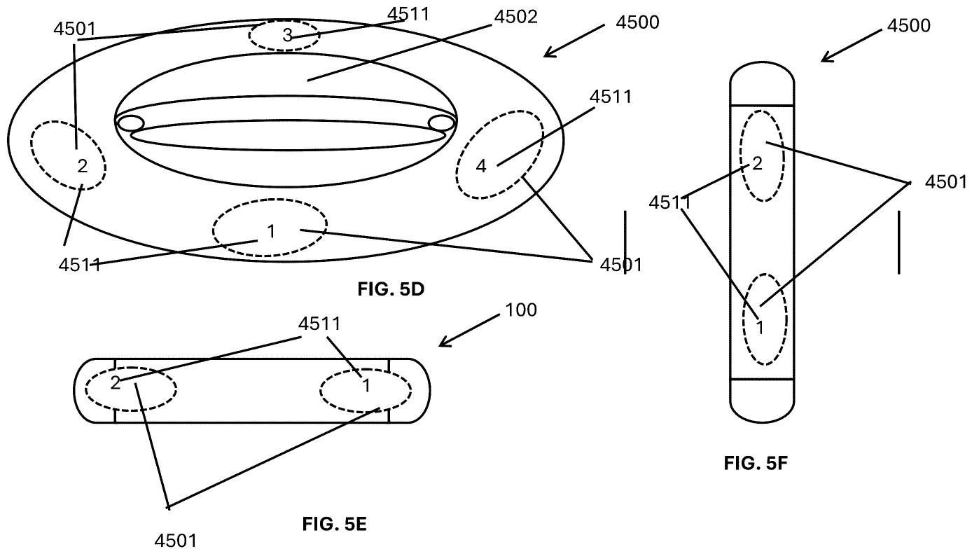

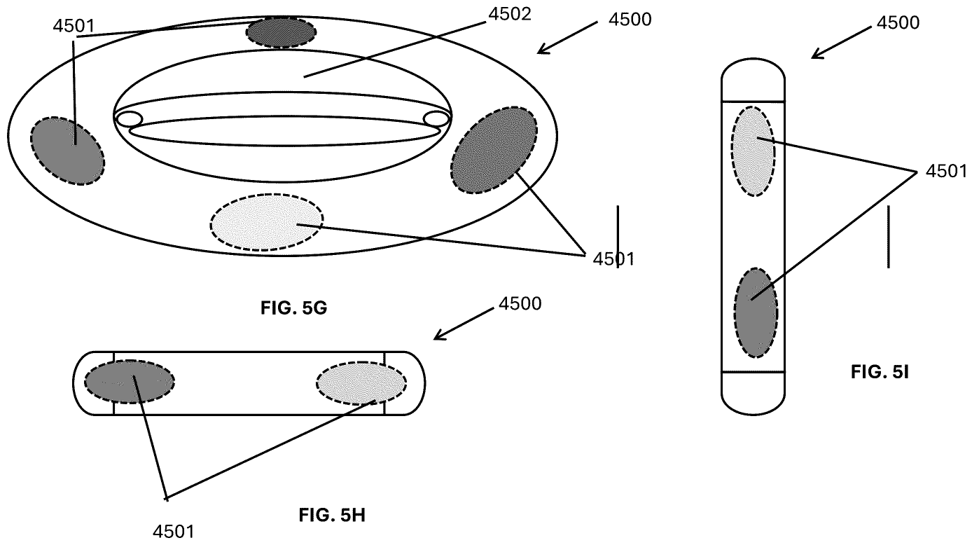

FIGS. 5A-5C are schematics of another prosthetic capsular device 4500 according to some embodiments herein. In some embodiments, the prosthetic capsular device 4500 may contain at least one chamber 4501. The at least one chamber 4501 can be used to contain pharmaceuticals for release into the eye. In further, embodiments, the at least one chamber 4501 is porous (e.g., through selection of a porous material or perforation or formation with small pores) such that the pharmaceuticals can be released into the eye through the holes in the at least one chamber 4501. In certain embodiments, the holes in the at least one chamber 4501 can range from 1 μm to 2 μm, although other sizes for the pores are also possible. In some embodiments the walls of the at least one chamber 4501 can be approximately 0.5 mm thick, although other thicknesses are also possible. In some embodiments the at least one chamber 4501 can be embossed with or otherwise include a mark 4511 such as a number or letter for future identification. In some embodiments, the at least one chamber 101 can have a variety of different color tints for future identification. In some embodiments, a port for docking 4810 can be coupled to each of the at least one chambers 4501.

In some embodiments, the device 4500 can have any number of chambers 4501, ranging from 1 chamber to 8 chambers (e.g., 2 chambers, 3 chambers, 4 chambers, 5 chambers, 6 chambers, and 7 chambers) or other numbers of chambers.

In some embodiments, the thickness of the walls of the chambers can range between 0.45 mm and 0.55 mm (e.g., 0.46 mm. 0.47 mm, 0.48 mm, 0.49 mm, 0.5 mm, 0.51 mm, 0.52 mm, 0.53 mm, and 0.54mm).

In some embodiments, the prosthetic capsular device 4500, may have an internal sidewall 4502 between the interior of the anterior portion and the interior of the posterior portion. In some embodiments the internal sidewall 4502 is convex. In some embodiments, the internal sidewall 4502 is concave. In some embodiments, the internal sidewall is flat. In some embodiments, the internal sidewall 4502 is porous or includes pores so as to diffuse a material held within a chamber 4501 through the sidewall 4502.

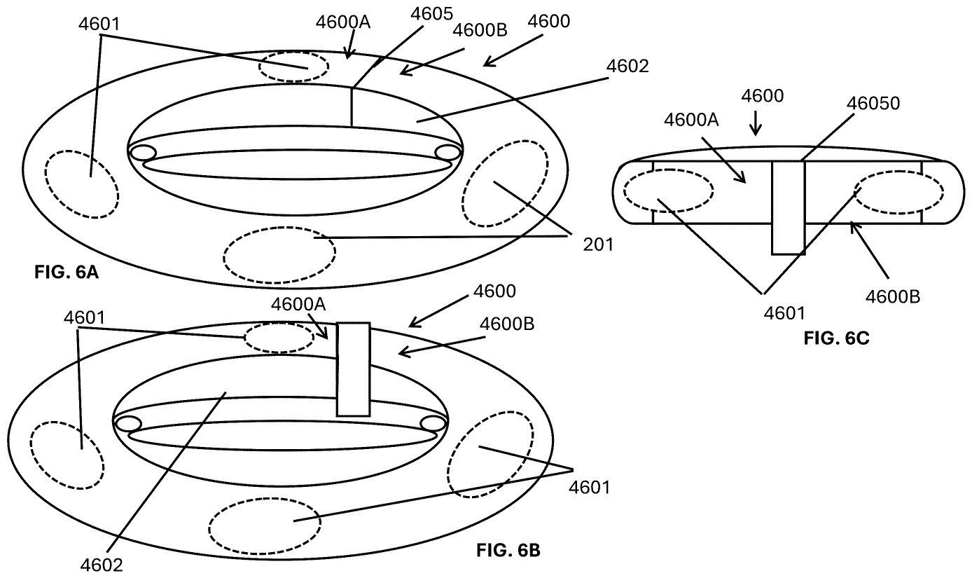

FIGS. 6A-6C demonstrate various perspective views of an example of a prosthetic capsular device 4600. In some embodiments, the device 4600 includes features described with respect to the devices described in U.S. Pat. No. 10,603,162 which is hereby incorporated by reference in its entirety, or modifications thereof.

In some embodiments, the device 4600 includes features described with in U.S. Pat. No. 9,358,103, which is hereby incorporated by reference in its entirety, or modifications thereof. For example, device 4600 can comprise an anterior side, a posterior side, and one or more sidewalls extending between the anterior side and the posterior side, a cavity or opening defined by the anterior side, posterior side, and the one or more sidewalls. Device 4600 can be configured to comprise or receive or more intraocular lenses, electronic devices, or other intraocular lenses, including, but not limited to: spherical, aspheric, wavefront, convex, concave, extended depth of focus, pinhole or small aperture, multifocal (diffractive, refractive, zonal), toric accommodative, ultraviolet (UV) filtering, diffractive chromatic aberration reducing lenes, light adjustable lenses (ultraviolet light adjustable, femtosecond phase wrapping), and optical powers ranging from any positive diopter value (e.g. including +35 D and above) to any negative diopter value (e.g. −35 and below)

In some embodiments, the device 4600 can comprise a break 4605 creating two distinct ends 4600A and 4600B as illustrated in FIGS. 6B-6C. In some embodiments the two distinct ends 4600A and 4600B can interlock such that the device 4600 takes a continuous shape as illustrated in FIG. 6A. In some embodiments, substantially the whole device 4600 can comprise silicone and/or a soft silicone polymer. In addition, in certain embodiments, substantially the whole device 4600 can comprise a flexible and/or elastic material. As such, the device 4600, can be foldable or collapsible for implantation into the eye through a small incision. Once inserted into the eye, the device 4600 can naturally unfold and self-expand into its expanded configuration as illustrated in FIG. 6A.

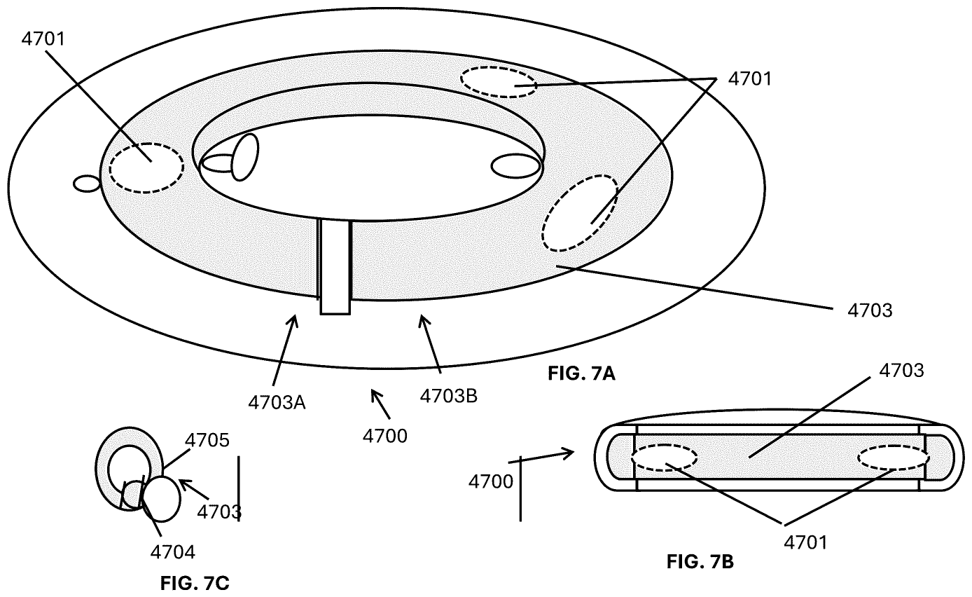

FIGS. 7A-7C demonstrate various perspective views of an example of a prosthetic capsular device. In some embodiments, the device 4700 includes features described with respect to the devices described in U.S. Pat. No. 8,900,300, U.S. Pat. No. 9,414,907, U.S. Pat. No. 9,358,103, U.S. Pat. No. 10,603,162 and/or any IOLs known to those skilled in the art. The prosthetic device 4700 may comprise any prosthetic device as described herein with reference to FIGS. 1-6C.

FIGS. 7A-7C are schematics of the flexible drug delivery device 4703 coupled to a prosthetic capsular device 4700. In some embodiments the flexible drug delivery device 4703 may have two distinct ends 4703A and 4703B. In some embodiments the drug delivery device may comprise at least one chamber 4701. In some embodiments, the flexible drug delivery device may comprise an upper lip 4705 and a lower lip 4704, such that applying pressure to the exterior of the lower lip causes it to fold inwards as illustrated by the cross section of the flexible drug delivery device 4703 in FIG. 7C.

In some embodiments, the at least one chamber 4701 can be used to contain pharmaceuticals for release into the eye. In further embodiments, the at least one chamber 4701 is porous such that the pharmaceuticals can be released into the eye though the holes in the at least one chamber 4701. In certain embodiments the holes in the at least one chamber can range from 1 μm to 2 μm. In some embodiments, the walls of the at least one chamber 4701 can be approximately 0.5 mm thick. In some embodiments, the at least one chamber 4701 can be embossed with a mark such as a number or letter for future identification. In some embodiments the at least one chamber 4701 can have a variety of different color tints for future identification

In some embodiments, the flexible drug eluting device 4703 can have any number of chambers ranging from 1 chamber to 8 chambers (e.g., 2 chambers, 3 chambers, 4 chambers, 5 chambers, 6 chambers, and 7 chambers), or other numbers of chambers.

In some embodiments, walls of the chambers can range in thickness from 0.45 mm and 0.55 mm (e.g., 0.46 mm, 0.47 mm, 0.48 mm, 0.49 mm, 0.5 mm, 0.51 mm, 0.52 mm, 0.53 mm, and 0.54 mm), as well as other wall thicknesses both thicker and thinner than the listed values.

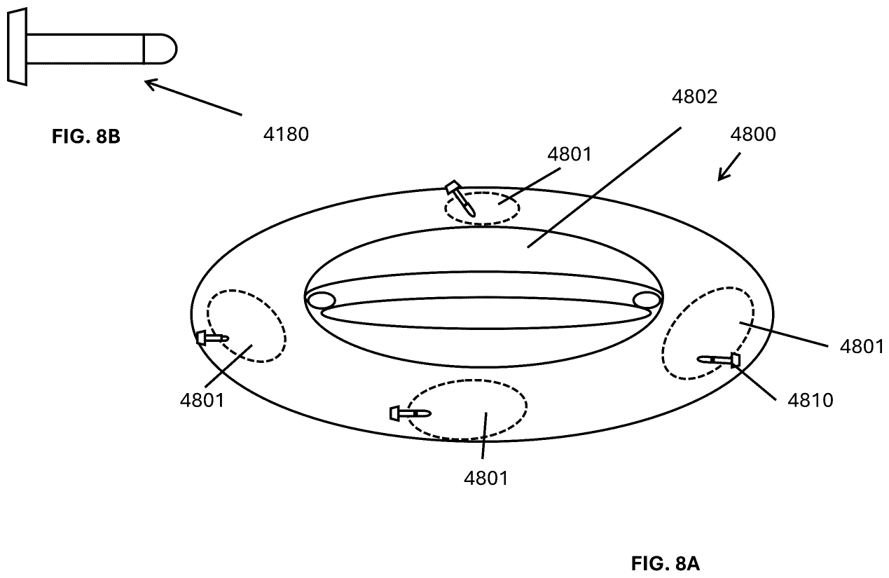

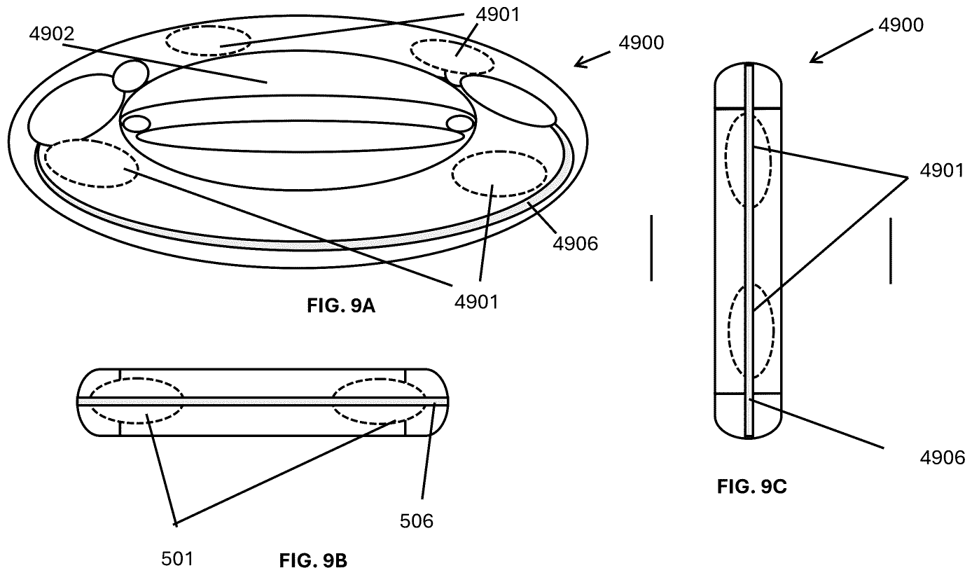

FIGS. 9A-9C demonstrate various perspective views of an example of a prosthetic capsular device. In some embodiments, the device 4900 includes features described with respect to devices described in the U.S. Pat. No. 10,603,162 which is hereby incorporated by reference in its entirety, or modifications thereof. The prosthetic device 4900 may comprise any prosthetic device as described herein with reference to FIGS. 1-8B.

In some embodiments, the device 4900 includes features described with respect to the devices described in U.S. Pat. No. 9,358,103, which is hereby incorporated by reference in its entirety, or modifications thereof. For example, the device 4900 can comprise an anterior side, a posterior side, and one or more sidewalls extending between the anterior side and the posterior side, a cavity or opening defined by the anterior side, posterior side, and the one or more sidewalls. The device 4900 can be configured to comprise or receive one or more intraocular lenses, electronic devices, or other intraocular devices held within the cavity. The IOLs may comprise any and all lens powers and designs that are currently known in the art of intraocular lenses, including, but not limited to: spherical, aspheric, wavefront, convex, concave, extended depth of focus, pinhole or small aperture, multifocal (diffractive, refractive, zonal), toric, accommodative, ultraviolet (UV) filtering, diffractive chromatic aberration reducing lenses, light adjustable lenses (ultraviolet light adjustable, femtosecond phase wrapping), and optical powers ranging from any positive diopter value (e.g. including +35 D and above) to any negative diopter value (e.g., including −35 and below).

In some embodiments, the device 4900 can comprise a fissure, opening, or slit 4906 around the perimeter of the lateral portion of the housing structure wherein the exterior surface of the lateral portion has access to chambers 4901 on the interior of the lateral portion.

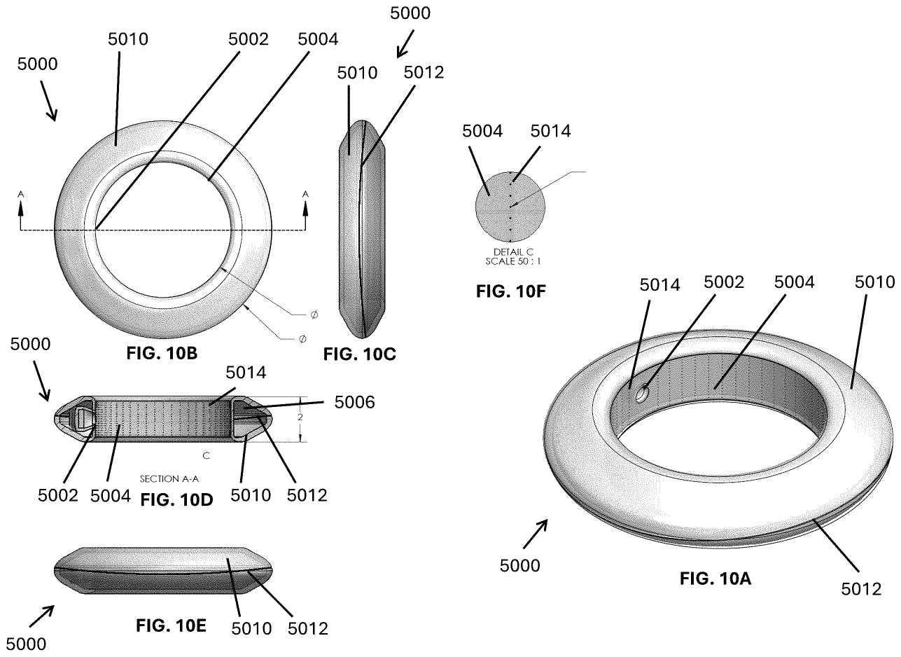

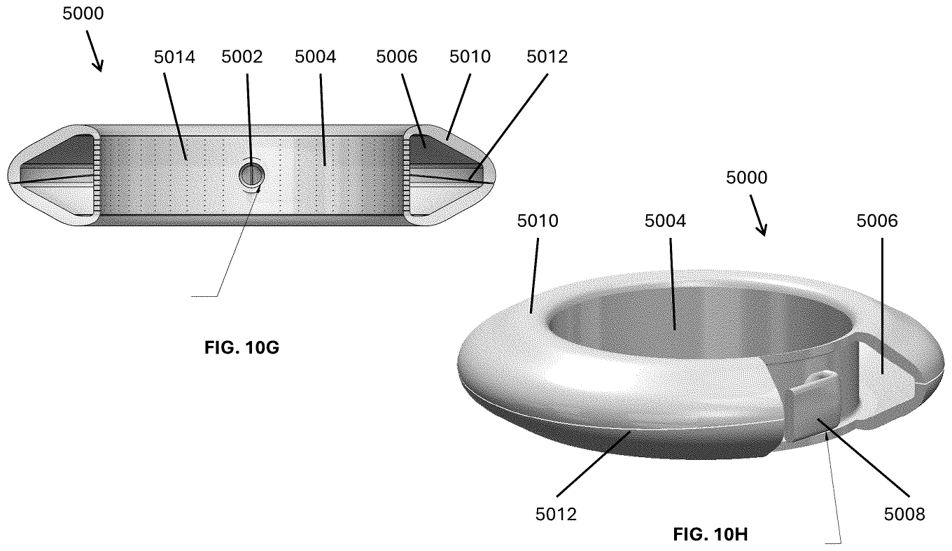

FIGS. 10A-10H illustrate various views of a device 5000. The device 5000 can be a prosthetic capsular device and can include, in some embodiments, any of the features described above with reference to the prior figures or throughout this application. As shown in the perspective view of FIG. 10A, the device 5000 is generally ring shaped and can include a profile as described previously and can also include additional features as shown in FIGS. 10A-10H and described here.

For example, as shown in the cross-sectional view of FIG. 10D (taken along the line A-A illustrated in the top view of FIG. 10B), the device 5000 can include an interior chamber 5006. The interior chamber 5006 can be configured to receive, in some embodiments, a pharmaceutical that can be diffused from the device 5000 into the eye, through, for example, the interior wall 5004 of the device 5000. To this end, as best shown in FIGS. 10A, 10D, 10F, and 10G, the interior wall 5004 of the device 5000 can be porous (e.g., including one or more pores 5014). The pores 5014 can be configured to allow material (e.g., a medicament or pharmaceutical housed within the interior chamber 5006 to diffuse through the interior wall.

As shown in FIGS. 10A, 10D, 10G, and 10H, the device 5000 can also include a port 5002, which, in the illustrated example, is formed on the interior wall 5004 of the device 5000. The port 5002 can be configured to allow material to be injected or otherwise filled into the interior cavity 5006. In the illustrated example, the port 5002 is shown as circular and is sized to allow entrance for a 27 gauge needle, although other sizes and are possible. As shown in the partial section view of FIG. 10H, the circular port 5002 can, within the cavity, transition to a flattened and/or kinked tube 5008 that, when the needles is retracted, seals the port 5002 keeping the medicant or pharmaceutical within the cavity 5006.

FIGS. 10A, 10C, 10D, 10E, 10G and 10H also show that than a lateral outsidewall 5010 of the device 5000 can include a slit, opening, or cut 5012. This can facilitate manufacturing as a mold can be included to create the internal cavity 5006. In some embodiments, the cut 5012 can be sealed by silicone or other adhesives. The shape of the cut 5012 can be wave like, for example, as shown, to aid in alignment and centering.

In some embodiments, the device 5000 can be configured to be inserted into a prosthetic device as described herein with reference to FIGS. 1-9C. In some embodiments, the device 5000 may comprise any prosthetic device as described herein with reference to FIGS. 1-9C.

In some embodiments, any of the prosthetic capsular devices, drug delivery capsules, flexible drug eluting devices, ports, chambers, or related intraocular components described herein can be formed in whole or in part from crosslinked polyisobutylene (xPIB) materials. xPIB materials can provide a combination of softness, flexibility, and elastic recovery suitable for intraocular implantation while maintaining dimensional stability of the device once implanted in the natural capsular bag. In certain embodiments, xPIB materials can exhibit high chemical inertness and non-reactivity with a wide range of intraocular drugs, including but not limited to small-molecule pharmaceuticals, biologics, anti-VEGF agents, corticosteroids, immunomodulators, and glaucoma medications. The non-reactive nature of xPIB can reduce or prevent undesirable interactions between the device material and the drug, such as adsorption, denaturation, degradation, or catalytic breakdown, thereby helping to preserve the potency, stability, and predictable release profile of the pharmaceutical over time. In some embodiments, one or more portions of the device, such as the lateral wall, interior chamber-defining walls, drug-eluting chambers, or port structures, can be formed from xPIB to maximize compatibility with the intraocular drugs stored or eluted from the device. In other embodiments, xPIB can be combined with other ophthalmically acceptable materials, such as silicone, acrylic, hydrogel, or Collamer, to tailor mechanical properties, permeability characteristics, and optical performance while maintaining the drug compatibility benefits afforded by the xPIB component. In some embodiments, xPIB regions can form the primary drug-contacting surfaces of chambers, cavities, or ports so that the intraocular drug is substantially exposed only to xPIB and the surrounding ocular media, thus reducing the risk of drug-material interactions that could otherwise alter drug concentration, cause particulate formation, or compromise patient safety.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A system for intraocular drug delivery, comprising:
a prosthetic capsular device configured to be inserted into a natural capsular bag of an eye after removal of a lens, the prosthetic capsular device comprising a housing structure having an anterior portion with an anterior opening, a posterior portion with a posterior opening, and a lateral portion interposed between the anterior portion and the posterior portion, the anterior portion, posterior portion, and lateral portion together defining an internal cavity; and
a drug delivery capsule configured to be received within the internal cavity of the prosthetic capsular device, the drug delivery capsule comprising a posterior surface and an anterior surface meeting at an outer end of the capsule, an interior surface having a shape of a section of a radial surface of a cylinder and being concave, a radial surface extending from an inner end to the outer end of the capsule, an inner cavity configured to receive a drug, one or more diffusion openings extending from an exterior surface of the capsule to the inner cavity and configured to permit the drug to elute from the inner cavity into the interior of the prosthetic capsular device, and a slit extending from the exterior surface of the capsule to the inner cavity and configured to permit introduction of the drug into the inner cavity,
wherein the drug delivery capsule is sized and shaped such that the concave interior surface of the capsule conforms at least in part to an interior surface of the housing structure when the capsule is disposed within the internal cavity, and wherein the prosthetic capsular device and the drug delivery capsule together are configured to position the drug in a location peripherally of a visual axis of the eye and to dispense the drug from within the natural capsular bag over an extended period of time.

2. The system of claim 1, wherein the prosthetic capsular device further comprises a posterior flange disposed on a posterior interior surface of the housing structure and an anterior flange disposed on an anterior interior surface of the housing structure, the posterior flange and the anterior flange extending toward one another to retain the drug delivery capsule within the internal cavity.

3. The system of claim 2, wherein the posterior flange and the anterior flange are configured to meet along a middle plane of the prosthetic capsular device.

4. The system of claim 2, wherein the posterior flange and the anterior flange are separated by a gap configured to permit insertion and removal of the drug delivery capsule.

5. The system of claim 1, wherein the one or more diffusion openings are disposed on the concave interior surface of the drug delivery capsule.

6. The system of claim 1, wherein the one or more diffusion openings are disposed on the radial surface of the drug delivery capsule.

7. The system of claim 1, wherein the slit is disposed on the concave interior surface of the drug delivery capsule and is configured to receive an instrument for injecting the drug into the inner cavity.

8. The system of claim 1, wherein the inner cavity of the drug delivery capsule is pre-filled with the drug during manufacture.

9. The system of claim 1, wherein the drug comprises at least one of a solid drug, a gel drug, a polymerized drug, or a liquid drug.

10. The system of claim 1, wherein the internal cavity of the prosthetic capsular device is configured to receive a plurality of said drug delivery capsules.

11. The system of claim 1, wherein at least a portion of the prosthetic capsular device, the drug delivery capsule, or both is formed from crosslinked polyisobutylene.

12. The system of claim 1, wherein the prosthetic capsular device is substantially symmetrical about a plane extending through a midpoint between the anterior portion and the posterior portion.

13. The system of claim 1, wherein the prosthetic capsular device is asymmetrical such that the anterior portion has a different shape, contour, angle, thickness, or radial profile than the posterior portion.

14. An intraocular drug delivery device, comprising:
an annular body having an interior wall and a lateral outsidewall, the annular body defining an interior chamber configured to contain a pharmaceutical;
wherein the interior wall comprises a plurality of pores extending therethrough and configured to permit the pharmaceutical to diffuse from the interior chamber into an eye; and
wherein the interior wall further comprises a fill port in fluid communication with the interior chamber and configured to receive the pharmaceutical.

15. The intraocular drug delivery device of claim 14, wherein the intraocular drug delivery device is configured to be inserted into a prosthetic capsular device.

16. The intraocular drug delivery device of claim 14, wherein the fill port is disposed on the interior wall and is circular.

17. The intraocular drug delivery device of claim 16, wherein the fill port is sized to receive a needle.

18. The intraocular drug delivery device of claim 14, wherein the lateral outside wall comprises a slit, opening, or cut extending from an exterior surface of the annular body to the interior chamber.

19. The intraocular drug delivery device of claim 18, wherein the slit, opening, or cut has a wave-like shape.

20. The intraocular drug delivery device of claim 18, wherein the slit, opening, or cut is sealed with silicone or an adhesive after formation of the interior chamber.
